(19) United States
(12) Patent Application Publication
(10) Pub. No.: US 2026/0132429 A1
(43) Pub. Date: May 14, 2026

(54) PROCESSES AND SYSTEMS FOR FERMENTATIVE ALCOHOL PRODUCTION AND RECOVERY

(71) Applicant: Gevo, Inc., Englewood, CO (US)

(72) Inventors: Kent Evans, Littleton, CO (US); Paul Bloom, Castle Rock, CO (US)

(21) Appl. No.: 19/386,105

(22) Filed: Nov. 11, 2025

Related U.S. Application Data
(60) Provisional application No. 63/719,570, filed on Nov. 12, 2024.

(51) Int. Cl. C12P 7/16; C10L 1/02; C12N 1/16; C12P 7/06; C12R 1/645

(52) U.S. Cl. CPC C12P 7/16; C10L 1/02; C12N 1/16; C12P 7/06; C10L 2200/0446; C10L 2200/0469; C12R 2001/645

(57) ABSTRACT

Disclosed are systems and methods for producing renewable alcohols by fermentative processes and recovering the product alcohols by extraction. The disclosure also provides processes and systems for converting renewable alcohols to transportation fuels or blending renewable alcohols with transportation fuels, such as gasoline, jet fuel, and diesel fuels.

### RELATED APPLICATIONS

This application claims the benefit of U.S. Provisional Patent Application No. 63/719,570 filed Nov. 12, 2024, the entire contents of which is incorporated by reference herein.

### FIELD

This disclosure relates to systems and methods for producing renewable alcohols by fermentative processes and recovering the product alcohols by extraction.

### BACKGROUND

Alcohols are important industrial chemicals, useful as reagents, solvents, fuel additives, feedstock chemicals in the plastics industry, and chemical intermediates. Renewable alcohols may be used in the production of transportation fuels, such as gasoline, jet fuel, and diesel fuels. Renewable alcohols have also been blended with transportation fuels, namely gasoline and diesel fuel, in an effort to reduce greenhouse gas emissions. Accordingly, there is a high demand for alcohols such as butanol for efficient and environmentally friendly methods of producing the renewable alcohols.

One such environmentally friendly production method is fermentation. In order to develop an economically competitive fermentation process, a number of factors, such as a) the development of a microorganism (“biocatalyst”) that may produce the alcohol, b) carbon sources capable of being metabolized by the microorganism, c) recovery of the alcohol from a fermentation broth, d) co-product formation, and e) the potential for contamination may all be considered in the development of this process. In particular, the production of alcohol using fermentation by a microorganism may be limited by the toxicity of the alcohol to the microorganism.

An option to address toxicity is the removal of the alcohol from the fermentation vessel as it is being produced. In situ product removal (ISPR) or extractive fermentation using liquid-liquid extraction, for example, may be used to remove alcohol from the fermentation vessel as it is produced, thereby allowing the microorganism to produce alcohol at higher yields. In liquid-liquid extraction, also known as solvent extraction, an extractant is contacted with the fermentation broth to partition the alcohol, e.g., butanol, between the fermentation broth and the extractant phase. An “extractant” may also be known as a “solvent,” and the terms may be used interchangeably. The alcohol, e.g., butanol, and the extractant or solvent are then recovered by a separation process, such as distillation. In the recovery process, the alcohol may also be separated from any water, non-condensable gas, and/or fermentation byproducts that may have been removed from the fermentation broth through use of the extractant.

Such extractive fermentation processes, including liquid-liquid extraction, may increase the complexity, capital costs, operating costs, and overall energy usage of the alcohol production process. For example, a viable liquid-liquid extraction process may require numerous parameters, including a) a particular amount of contact between an extractant and a fermentation broth for efficient mass transfer of the product alcohol into the extractant, b) good phase separation of the extractant from the fermentation broth (during and/or after fermentation), c) efficient recovery and/or recycling of the extractant, d) minimal degradation of the ability of the extractant to extract the product alcohol (e.g., by inhibiting the lowering of the partition coefficient for the product alcohol into the extractant), and/or e) minimal contamination of the extractant by lipids that lower the partition coefficient over a long-term operation.

Various liquid-liquid extraction processes have been described for partitioning a product alcohol, e.g., butanol, between a fermentation broth and an extractant phase. Methods for producing and recovering butanol from a fermentation broth may comprise steps of contacting the fermentation broth with a water immiscible organic extractant to form a two-phase mixture comprising an aqueous phase and a butanol-containing organic phase. In many such cases, the extractant may be a C12 to C22 fatty alcohol, a C12 to C22 fatty acid, an ester of a C12 to C22 fatty acid, a C12 to C22 fatty aldehyde, or a mixture thereof. The water-immiscible organic extractant may comprise one or more of a C12 to C22 fatty alcohol, a C12 to C22 fatty acid, an ester of a C12 to C22 fatty acid, and/or a C12 to C22 fatty aldehyde in combination with one or more of a C7 to C11 alcohol, a C7 to C11 carboxylic acid, an ester of a C7 to C11 carboxylic acid, and/or a C7 to C11 aldehyde.

Thus, there is a continuing need for efficient and economical systems and processes for separating a product alcohol, e.g., butanol, from a fermentation broth to address the challenge of alcohol toxicity to microorganisms used for microbial fermentation. There is also a need to develop more efficient methods and systems for producing alcohols that may be blended with transportation fuels.

### SUMMARY

This disclosure describes, among other things, methods of producing renewable alcohol and/or renewable fuels or fuel blends comprising diesel fuel and renewable alcohol, particularly butanol, as well as methods for extracting and recovering the renewable alcohol and converting the renewable alcohol to fuel. The disclosure also describes renewable fuels or fuel blends comprising diesel fuel and renewable alcohol.

In some embodiments, a method for producing renewable alcohol includes: a) providing a recombinant yeast cell comprising an alcohol-producing metabolic pathway; b) mixing the recombinant yeast cell with a source of glucose to produce a reaction mixture and fermenting the reaction mixture to produce alcohol; c) contacting the reaction mixture and the alcohol with an extractant comprising diesel fuel, where the alcohol partitions into a non-aqueous organic extractant phase, and the reaction mixture partitions into an aqueous phase; and d) distilling the non-aqueous organic extractant phase to separate the alcohol.

In some embodiments, a method for producing renewable fuel includes: a) providing a recombinant yeast cell comprising an alcohol-producing metabolic pathway; b) mixing the recombinant yeast cell with a source of glucose to produce a reaction mixture and fermenting the reaction mixture to produce alcohol; c) contacting the reaction mixture and the alcohol with an extractant comprising diesel fuel to produce a non-aqueous organic extractant phase and an aqueous phase, where the alcohol partitions into the non-aqueous organic extractant phase, and the reaction mixture partitions into the aqueous phase; d) separating the non-aqueous organic extractant phase to form the renewable fuel; and, optionally, e) recycling the aqueous phase to a fermenter.

In some embodiments, the reaction mixture and the alcohol are contacted with the extractant before the concentration of the alcohol reaches a level toxic to the recombinant yeast cell.

In some embodiments, the diesel fuel comprises fossil diesel, petroleum diesel, synthetic diesel, biodiesel, renewable diesel, hydrogenated oils, hydrogenated fats, or a combination thereof.

In some embodiments, the alcohol producing the metabolic pathway of step (a) may comprise an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.

In some embodiments, the recombinant yeast cell may be selected from the group consisting of Saccharomyces, Kluyveromyces, Candida, Pichia, Issatchenkia, Debaryomyces, Hansenula, Yarrowia, Schizosaccharomyces, and combinations thereof.

In some embodiments, the renewable alcohol may be selected from the group consisting of ethanol, 1-butanol, 2-butanol, isobutanol, tert-butanol, and combinations thereof.

A multiphase fermentation composition including a) an aqueous phase comprising a recombinant yeast cell comprising an isobutanol-producing metabolic pathway and, optionally, a source of glucose; and b) a non-aqueous organic extractant phase comprising isobutanol and diesel fuel.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter of this disclosure are contemplated as being part of the embodiments disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

FIG. 1 schematically illustrates an embodiment of the methods and systems of the disclosure, in which an extractant is contacted with an alcohol-rich fermentation broth, forming an aqueous phase containing an alcohol-reduced broth and an organic phase containing a blend of the extractant and the alcohol.

FIG. 2 illustrates an embodiment of the methods and system of the disclosure, in which a stream of an extractant composition is injected into the bottom of a liquid-liquid extraction unit, and a stream of the fermentation broth is injected into the top of the liquid-liquid extraction unit directly from a fermentation vessel.

FIG. 3 is a graph depicting the effect of different organic extractants or solvents on the isobutanol yield of batch fermentation.

FIG. 4 is a graph depicting the effect of the usage of fresh and regenerated biodiesel on the isobutanol yield of batch fermentation.

FIG. 5 is a graph depicting the effect of repeated fermentations on isobutanol removal from biodiesel.

### DETAILED DESCRIPTION

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

In order to further define this invention, the following terms and definitions are herein provided.

Reference throughout this specification to “some embodiments,” “one embodiment” or “an embodiment” means a particular feature, structure or characteristic described in connection with the embodiment is included in at least one embodiment. Thus, the appearances of the phrases “in some embodiments,” “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the context clearly dictates otherwise.

The word “about” when immediately preceding a numerical value means a range of plus or minus 10% of that value, e.g., “about 50” means 45 to 55, “about 25,000” means 22,500 to 27,500, etc. Furthermore, the phrases “less than about” a value or “greater than about” a value should be understood in view of the definition of the term “about” provided herein.

As used herein, “recombinant microorganism” refers to a microorganism, such as bacterium or yeast, that is modified by use of recombinant DNA techniques, for example, by engineering a host cell to comprise a biosynthetic pathway, such as a biosynthetic pathway to produce an alcohol (e.g., butanol).

As used herein, “fermentable carbon source” refers to a carbon source capable of being metabolized by the microorganisms disclosed herein for the production of alcohol. Suitable fermentable carbon sources include, but are not limited to, monosaccharides such as glucose or fructose, disaccharides such as lactose or sucrose, oligosaccharides, polysaccharides such as starch or cellulose, C₅ sugars such as xylose and arabinose, carbon substrates such as methane, and mixtures thereof. The carbon source may be derived from biomass. The terms “fermentable carbon source,” “carbon substrate,” and “fermentable carbon substrate” are used interchangeably.

As used herein, the term “feedstock” refers to a feed in a fermentation process, the feed containing a fermentable carbon source with or without undissolved solids, and where applicable, the feed containing the fermentable carbon source before or after the fermentable carbon source has been liberated from starch or obtained from the breakdown of complex sugars by further processing, such as by liquefaction, saccharification, or other processes. Feedstock includes or may be derived from biomass. Suitable feedstocks include, but are not limited to, rye, wheat, barley, corn, corn mash, cane, cane mash, cellulosic material, lignocellulosic material, and mixtures thereof.

The terms “fermentation broth,” “fermentation liquid,” “fermentation medium,” and “fermented mixture” are used interchangeably and refer to a mixture containing, among other constituents, fermentative microorganisms, fermentable carbon sources (e.g., glucose), and alcohol.

As used herein, the term “diesel fuel(s)” include, but is not limited to, fossil diesel, petroleum diesel, synthetic diesel, biodiesel, renewable diesel, hydrogenated oils and fats, and blends of the diesel fuels. This disclosure is not intended to be limited to any particular type of diesel fuel.

As used herein, the term “greenhouse gas emissions” refers to compounds or components of vehicular exhaust or exhaust from other diesel combustion engine emissions which include, but are not limited to, carbon dioxide (CO₂), nitrous oxide (N₂O), and methane (CH₄).

As used herein, the term “diesel combustion engine” refers to diesel engines, including heavy duty diesel engines, that generate motive power by the burning of diesel with air inside the engine, the hot gases produced being used to drive a piston or do other work as they expand. Such diesel engines include but are not limited to vehicle diesel engines including, but not limited to, automotive, marine and aviation engines.

Disclosed herein is a process for producing renewable alcohol using microbial fermentation. Renewable alcohols include, but are not limited to, C₁ to C₈ alkyl alcohols, isomers of C₁ to C₈ alkyl alcohols, or mixtures thereof. In some embodiments, the alcohols are C₂ to C₈ alkyl alcohols. In some embodiments, the alcohols are C₂ to C₅ alkyl alcohols or C₃ to C₆ alkyl alcohols. It will be appreciated that C₁ to C₈ alkyl alcohols include, but are not limited to, methanol, ethanol, propanol, butanol, pentanol, and hexanol. C₂ to C₈ alkyl alcohols include, but are not limited to, ethanol, propanol, butanol, and pentanol.

In order to develop an economically viable microbial fermentation process, a number of factors, such as the development or identification of a microorganism (“biocatalyst”) that may produce the alcohol, the identification of carbon sources capable of being metabolized by the microorganism, the efficient recovery of the alcohol from a fermentation broth, co-product formation, and the potential for contamination, may be considered.

### Fermentative Recombinant Microorganism

The process for producing renewable alcohol described herein may use any alcohol-producing microorganism, including recombinant alcohol-producing microorganisms. Though many native or naturally occurring alcohol-producing microorganisms (e.g., bacteria, yeast) exist, such native microorganisms may not produce alcohol at a high enough yield for commercialization. For example, Clostridium acetobutylicum and other Clostridia are known to produce butanol via fermentation. In fact, acetone-butanol-ethanol (ABE) fermentation by Clostridium acetobutylicum is one of the oldest known industrial fermentations (as are also the pathways and genes responsible for the production of the acetone, butanol, and ethanol). Production of butanol by the ABE process, however, is limited by the toxic effect of the butanol on Clostridium acetobutylicum and by the generation of coproducts, namely acetone and ethanol. Genetic modification using known molecular biological techniques may be used to develop recombinant microorganisms that fermentatively produce alcohol, including butanol, at higher yields. Suitable microorganisms that may be genetically modified include bacteria, cyanobacteria, filamentous fungi, or yeast.

Recombinant microorganisms may be engineered to express a selected metabolic pathway and/or to produce a desired metabolite, such as alcohol, to reduce or eliminate the production of undesired coproducts, and/or to otherwise increase the yield of the desired metabolite, such as alcohol (e.g., butanol). Metabolites include starting materials (e.g., glucose or pyruvate), intermediates (e.g., 2-ketoisovalerate, acetaldehyde), and end products (e.g., ethanol, butanol, other alcohols) of a selected metabolic pathway. Metabolites may be used to construct more complex molecules or be broken down into simpler molecules. Intermediate metabolites may be synthesized from other metabolites, used to make more complex molecules, or broken down into simpler molecules, often with the release of chemical energy. Examples of metabolites include glucose, pyruvate, and alcohol, such as butanol.

Recombinant microorganisms that produce alcohol are known in the art (e.g., Ohta et al., Appl. Environ. Microbiol. 57:893-900 (1991); Underwood et al., Appl. Environ. Microbiol. 68:1071-81 (2002); Shen and Liao, Metab. Eng. 10:312-20 (2008); Hahnai et al., Appl. Environ. Microbiol. 73:7814-8 (2007); U.S. Pat. Nos. 5,514,583; 5,712,133; International Pat. Pub. No. WO 1995/028476; Feldmann et al., Appl. Microbiol. Biotechnol. 38:354-61 (1992); Zhang et al., Science 267:240-3 (1995); U.S. Pat. Pub. No. 2007/0031918A1; U.S. Pat. Nos. 7,223,575; 7,741,119; U.S. Pat. Pub. No. 2009/0203099A1; U.S. Pat. Pub. No. 2009/0246846A1; and International Pat. Pub. No. WO 2010/075241), which are herein incorporated by reference.

For example, the metabolic pathways of microorganisms may be genetically modified to produce butanol. The metabolite butanol may be produced by a recombinant microorganism metabolically engineered to express or over-express a metabolic pathway that converts pyruvate to butanol. The metabolic pathway may also be modified to reduce or eliminate undesired metabolites or coproducts, thereby improving the yield of the alcohol. The production of butanol by a recombinant microorganism is disclosed in, for example, U.S. Pat. Nos. 7,851,188; 7,993,889; 8,178,328; 8,206,970; and 9,790,521; and U.S. Pat. Pub. Nos. 2007/0292927; 2008/0182308; 2008/0274525; 2009/0305363; 2009/0305370; 2011/0250610; 2011/0313206; 2011/0111472; and 2012/0258873, which are herein incorporated by reference. Recombinant microorganisms that produce butanol at higher yields are disclosed in U.S. Pat. No. 8,455,239 and International Pat. Pub. No. WO 2010/05125, which are herein incorporated by reference.

In some embodiments, the recombinant microorganism comprises a butanol biosynthetic pathway or a biosynthetic pathway for butanol isomers, such as 1-butanol, 2-butanol, or isobutanol. In some embodiments, the biosynthetic pathway converts pyruvate to a fermentative product. In some embodiments, the biosynthetic pathway converts pyruvate as well as amino acids to a fermentative product. In some embodiments, at least one, at least two, at least three, or at least four polypeptides catalyzing substrate to product conversions of a pathway are encoded by heterologous polynucleotides in the microorganism. In some embodiments, all polypeptides catalyzing substrate to product conversions of a pathway are encoded by heterologous polynucleotides in the microorganism.

Suitable microorganisms capable of producing alcohol (e.g., butanol) via a biosynthetic pathway include members of the genera Clostridium, Zymomonas, Escherichia, Salmonella, Serratia, Erwinia, Klebsiella, Shigella, Rhodococcus, Pseudomonas, Bacillus, Lactobacillus, Enterococcus, Alcaligenes, Paenibacillus, Arthrobacter, Corynebacterium, Brevibacterium, Schizosaccharomyces, Kluveromyces, Yarrowia, Pichia, Zygosaccharomyces, Debaryomyces, Candida, Brettanomyces, Pachysolen, Hansenula, Issatchenkia, Trichosporon, Yamadazyma, or Saccharomyces. In some embodiments, the recombinant microorganisms may be selected from the group consisting of Escherichia coli, Alcaligenes eutrophus, Bacillus lichenformis, Paenibacillus macerans, Rhodocuccus erythropolis, Pseudomonas putida, Lactobacillus plantarum, Enterococcus faecium, Enterococcus gallinarium, Enterococcus faecalis, Bacillus subtilis, Candida sonorensis, Candida methanosorbosa, Kluyveromyces lactis, Kluyveromyces marxianus, Kluveromyces thermotolerans, Issatchenkia orientalis, Debaryomyces hansenii, and Saccharomyces cerevisiae.

In some embodiments, the genetically modified microorganism is a recombinant yeast. In some embodiments, the genetically modified microorganism is a crabtree-positive recombinant yeast. Suitable yeasts include Saccharomyces, Zygosaccharomyces, Schizosaccharomyces, Dekkera, Torulopsis, Brettanomyces, and some species of Candida. Species of crabtree-positive yeast include, but are not limited to, Saccharomyces cerevisiae, Saccharomyces kluyveri, Schizosaccharomyces pombe, Saccharomyces bayanus, Saccharomyces mikitae, Saccharomyces paradoxus, Saccharomyces uvarum, Saccharomyces castelli, Zygosaccharomyces rouxii, Zygosaccharomyces bailli, and Candida glabrata.

In some embodiments, the host cell is Saccharomyces cerevisiae. Saccharomyces cerevisiae is known in the art and available from a variety of sources including, but not limited to, American Type Culture Collection (Rockville, Md.), Centraalbureau voor Schimmelcultures (CBS) Fungal Biodiversity Centre, LeSaffre, Gert Strand AB, Ferm Solutions, North American Bioproducts, Martrex, and Lallemand. S. cerevisiae may be available from a variety of sources including, but not limited to, BY4741, CEN.PK 113-7D, Ethanol Red® yeast, Ferm Pro™ yeast, Bio-Ferm® XR yeast, Gert Strand Prestige Batch Turbo alcohol yeast, Gert Strand Pot Distillers yeast, Gert Strand Distillers Turbo yeast, FerMax™ Green yeast, FerMax™ Gold yeast, Thermosacc® yeast, BG-1, PE-2, CAT-1, CBS7959, CBS7960, and CBS7961.

A recombinant host cell comprising an “engineered alcohol production pathway” (such as an engineered butanol or isobutanol production pathway) refers to a host cell containing a modified pathway that produces alcohol in a manner different than that normally present in the host cell. Such differences include production of an alcohol not typically produced by the host cell or increased or more efficient production.

Biosynthetic pathways for the production of isobutanol (IBA) that may be used include those described in U.S. Pat. Nos. 7,851,188; 7,993,388; and International Pat. Pub. No. WO 2007/050671, which are incorporated herein by reference. In some embodiments, the isobutanol biosynthetic pathway may comprise the following substrate-to-product conversions:
a) pyruvate to acetolactate, which may be catalyzed by, for example, acetolactate synthase;
b) the acetolactate from step a) to 2,3-dihydroxyisovalerate, which may be catalyzed by, for example, acetohydroxy acid reductoisomerase;
c) the 2,3-dihydroxyisovalerate from step b) to α-ketoisovalerate, which may be catalyzed by, for example, acetohydroxy acid dehydratase;
d) the α-ketoisovalerate from step c) to isobutyraldehyde, which may be catalyzed by, for example, a branched-chain α-keto acid decarboxylase; and,
e) the isobutyraldehyde from step d) to isobutanol, which may be catalyzed by, for example, a branched-chain alcohol dehydrogenase.

In some embodiments, the isobutanol biosynthetic pathway may comprise the following substrate to product conversions:
a) pyruvate to acetolactate, which may be catalyzed by, for example, acetolactate synthase;
b) the acetolactate from step a) to 2,3-dihydroxyisovalerate, which may be catalyzed by, for example, ketol-acid reductoisomerase;
c) the 2,3-dihydroxyisovalerate from step b) to α-ketoisovalerate, which may be catalyzed by, for example, dihydroxyacid dehydratase;
d) the α-ketoisovalerate from step c) to valine, which may be catalyzed by, for example, transaminase or valine dehydrogenase;
e) the valine from step d) to isobutylamine, which may be catalyzed by, for example, valine decarboxylase;
f) the isobutylamine from step e) to isobutyraldehyde, which may be catalyzed by, for example, omega transaminase; and,
g) the isobutyraldehyde from step f) to isobutanol, which may be catalyzed by, for example, a branched-chain alcohol dehydrogenase.

In some embodiments, the isobutanol biosynthetic pathway may comprise the following substrate to product conversions:
a) pyruvate to acetolactate, which may be catalyzed by, for example, acetolactate synthase;
b) the acetolactate from step a) to 2,3-dihydroxyisovalerate, which may be catalyzed by, for example, acetohydroxy acid reductoisomerase;
c) the 2,3-dihydroxyisovalerate from step b) to α-ketoisovalerate, which may be catalyzed by, for example, acetohydroxy acid dehydratase;
d) the α-ketoisovalerate from step c) to isobutyryl-CoA, which may be catalyzed by, for example, branched-chain keto acid dehydrogenase;
e) the isobutyryl-CoA from step d) to isobutyraldehyde, which may be catalyzed by, for example, acylating aldehyde dehydrogenase; and,
f) the isobutyraldehyde from step e) to isobutanol, which may be catalyzed by, for example, a branched-chain alcohol dehydrogenase.

Biosynthetic pathways for the production of 1-butanol that may be used include those described in U.S. Pat. Pub. No. 2008/0182308 and International Pat. Pub. No. WO 2007/041269, which are incorporated herein by reference. In some embodiments, the 1-butanol biosynthetic pathway may comprise the following substrate to product conversions:
a) acetyl-CoA to acetoacetyl-CoA, which may be catalyzed by, for example, acetyl-CoA acetyltransferase;
b) the acetoacetyl-CoA from step a) to 3-hydroxybutyryl-CoA, which may be catalyzed by, for example, 3-hydroxybutyryl-CoA dehydrogenase;
c) the 3-hydroxybutyryl-CoA from step b) to crotonyl-CoA, which may be catalyzed by, for example, crotonase;
d) the crotonyl-CoA from step c) to butyryl-CoA, which may be catalyzed by, for example, butyryl-CoA dehydrogenase;
e) the butyryl-CoA from step d) to butyraldehyde, which may be catalyzed by, for example, butyraldehyde dehydrogenase; and,
f) the butyraldehyde from step e) to 1-butanol, which may be catalyzed by, for example, butanol dehydrogenase.

Biosynthetic pathways for the production of 2-butanol that may be used include those described in U.S. Pat. No. 8,206,970; U.S. Pat. Pub. Nos. 2007/0292927 and 2009/0155870; and International Pat. Pub. Nos. WO 2007/130518 and WO 2007/130521, which are incorporated herein by reference. In some embodiments, the 2-butanol biosynthetic pathway may comprise the following substrate to product conversions:
a) pyruvate to alpha-acetolactate, which may be catalyzed by, for example, acetolactate synthase;
b) the alpha-acetolactate from step a) to acetoin, which may be catalyzed by, for example, acetolactate decarboxylase;
c) the acetoin from step b) to 3-amino-2-butanol, which may be catalyzed by, for example, acetonin aminase;
d) the 3-amino-2-butanol from step c) to 3-amino-2-butanol phosphate, which may be catalyzed by, for example, aminobutanol kinase;
e) the 3-amino-2-butanol phosphate from step d) to 2-butanone, which may be catalyzed by, for example, aminobutanol phosphate phosphorylase; and,
f) the 2-butanone from step e) to 2-butanol, which may be catalyzed by, for example, butanol dehydrogenase.

In some embodiments, the 2-butanol biosynthetic pathway may comprise the following substrate to product conversions:
a) pyruvate to alpha-acetolactate, which may be catalyzed by, for example, acetolactate synthase;
b) the alpha-acetolactate from step a) to acetoin, which may be catalyzed by, for example, acetolactate decarboxylase;
c) the acetoin to 2,3-butanediol from step b), which may be catalyzed by, for example, butanediol dehydrogenase;
d) the 2,3-butanediol from step c) to 2-butanone, which may be catalyzed by, for example, dial dehydratase; and,
e) the 2-butanone from step d) to 2-butanol, which may be catalyzed by, for example, butanol dehydrogenase.

Biosynthetic pathways for the production of 2-butanone that may be used include those described in U.S. Pat. No. 8,206,970 and U.S. Pat. Pub. Nos. 2007/0292927 and 2009/0155870, which are incorporated herein by reference. In some embodiments, the 2-butanone biosynthetic pathway may comprise the following substrate to product conversions:
a) pyruvate to alpha-acetolactate, which may be catalyzed by, for example, acetolactate synthase;
b) the alpha-acetolactate from step a) to acetoin, which may be catalyzed by, for example, acetolactate decarboxylase;
c) the acetoin from step b) to 3-amino-2-butanol, which may be catalyzed by, for example, acetonin aminase;
d) the 3-amino-2-butanol from step c) to 3-amino-2-butanol phosphate, which may be catalyzed by, for example, aminobutanol kinase; and,
e) the 3-amino-2-butanol phosphate from step d) to 2-butanone, which may be catalyzed by, for example, aminobutanol phosphate phosphorylase.

In some embodiments, the 2-butanone biosynthetic pathway may comprise the following substrate to product conversions:
a) pyruvate to alpha-acetolactate, which may be catalyzed by, for example, acetolactate synthase;
b) the alpha-acetolactate from step a) to acetoin which may be catalyzed by, for example, acetolactate decarboxylase;
c) the acetoin from step b) to 2,3-butanediol, which may be catalyzed by, for example, butanediol dehydrogenase;
d) the 2,3-butanediol from step c) to 2-butanone, which may be catalyzed by, for example, diol dehydratase.

The terms “acetohydroxyacid synthase,” “acetolactate synthase,” and “acetolactate synthetase” (abbreviated “ALS”) are used interchangeably herein to refer to an enzyme that catalyzes the conversion of pyruvate to acetolactate and CO₂. Example acetolactate synthases are known by the EC number 2.2.1.6 (Enzyme Nomenclature 1992, Academic Press, San Diego). These enzymes are available from a number of sources, including, but not limited to, Bacillus subtilis (GenBank Nos: CAB07802.1, Z99122, NCBI (National Center for Biotechnology Information) amino acid sequence, NCBI nucleotide sequence, respectively), CAB15618, Klebsiella pneumoniae (GenBank Nos: AAA25079, M73842), and Lactococcus lactis (GenBank Nos: AAA25161, L16975).

The term “ketol-acid reductoisomerase” (“KARI”), “acetohydroxy acid isomeroreductase,” and “acetohydroxy acid reductoisomerase” are used interchangeably and refer to enzymes capable of catalyzing the reaction of (S)-acetolactate to 2,3-dihydroxyisovalerate. Example KARI enzymes may be classified as EC number EC 1.1.1.86 (Enzyme Nomenclature 1992, Academic Press, San Diego) and are available from a vast array of microorganisms, including, but not limited to, Escherichia coli (GenBank Nos: NP_418222, NC_000913), Saccharomyces cerevisiae (GenBank Nos: NP_013459, NC_001144), Methanococcus maripaludis (GenBank Nos: CAF30210, BX957220), Bacillus subtilis (GenBank Nos: CAB14789, Z99118), and Anaerostipes caccae. Ketol-acid reductoisomerase (KARI) enzymes are described in U.S. Pat. Nos. 7,910,342; 8,129,162; and 9,512,408; U.S. Patent Application Pub. Nos. 2008/0261230; 2009/0163376; 2010/0197519; and International Pub. Nos. WO 2011/041415 and WO 2012/129555, which are incorporated herein by reference. Examples of suitable KARIs include those from Lactococcus lactis, Vibrio cholera, Pseudomonas aeruginosa PAO1, and Pseudomonas fluorescens PF5 mutants. In some embodiments, the KARI uses NADH or NADPH.

The terms “acetohydroxy acid dehydratase” and “dihydroxyacid dehydratase” (“DHAD”) refer to an enzyme that catalyzes the conversion of 2,3-dihydroxyisovalerate to α-ketoisovalerate. Example acetohydroxy acid dehydratases are known by the EC number 4.2.1.9. Such enzymes are available from a vast array of microorganisms, including, but not limited to, E. coli (GenBank Nos: YP_026248, NC000913), Saccharomyces cerevisiae (GenBank Nos: NP_012550, NC_001142), M. maripaludis (GenBank Nos: CAF29874, BX957219), B. subtilis (GenBank Nos: CAB14105, Z99115), L. lactis, and N. crassa. U.S. Pat. Pub. No. 2010/0081154; U.S. Pat. Nos. 7,851,188; and 8,241,878, which are incorporated herein by reference in their entireties, describe dihydroxyacid dehydratases (DHADs), including a DHAD from Streptococcus mutans and variants thereof.

The term “branched-chain α-keto acid decarboxylase,” “α-ketoacid decarboxylase,” “α-ketoisovalerate decarboxylase,” or “2-ketoisovalerate decarboxylase” (“KIVD”) refers to an enzyme that catalyzes the conversion of α-ketoisovalerate to isobutyraldehyde and CO₂. Example branched-chain α-keto acid decarboxylases are known by the EC number 4.1.1.72 and are available from a number of sources, including, but not limited to, Lactococcus lactis (GenBank Nos: AAS49166, AY548760; CAG34226, AJ746364), Salmonella typhimurium (GenBank Nos: NP_461346, NC_003197), Clostridium acetobutylicum (GenBank Nos: NP_149189, NC_001988), M. caseolyticus, and L. grayi.

The term “branched-chain alcohol dehydrogenase” (“ADH”) refers to an enzyme that catalyzes the conversion of isobutyraldehyde to isobutanol. Example branched-chain alcohol dehydrogenases are known by the EC number 1.1.1.265 but may also be classified under other alcohol dehydrogenases (specifically, EC 1.1.1.1 or 1.1.1.2). Alcohol dehydrogenases may be NADPH-dependent or NADH-dependent. Such enzymes are available from a number of sources, including, but not limited to, S. cerevisiae (GenBank Nos: NP_010656, NC_001136, NP_014051, NC_001145), E. coli (GenBank Nos: NP_417484, NC_000913), and C. acetobutylicum (GenBank Nos: NP_349892, NC_003030; NP_349891, NC_003030). U.S. Pat. Pub. No. 2009/0269823, which is incorporated herein by reference in its entirety, describes SadB, an alcohol dehydrogenase (ADH) from Achromobacter xylosoxidans. Alcohol dehydrogenases may also include horse liver ADH and Beijerinkia indica ADH, as described by U.S. Pat. Pub. No. 2011/0269199, which is incorporated herein by reference in its entirety.

The term “butanol dehydrogenase” refers to a polypeptide (or polypeptides) having enzyme activity that catalyzes the conversion of isobutyraldehyde to isobutanol or the conversion of 2-butanone and 2-butanol. Butanol dehydrogenases are a subset of a broad family of alcohol dehydrogenases. Butanol dehydrogenase may be NAD-dependent or NADP-dependent. The NAD-dependent enzymes are known as EC 1.1.1.1 and are available from, for example, Rhodococcus ruber (GenBank Nos: CAD36475, AJ491307). The NADP-dependent enzymes are known as EC 1.1.1.2 and are available from, for example, Pyrococcus furiosus (GenBank Nos: AAC25556, AF013169). Additionally, a butanol dehydrogenase is available from E. coli (GenBank Nos: NP_417484, NC_000913) and a cyclohexanol dehydrogenase is available from Acinetobacter species (GenBank Nos: AAG10026, AF282240). The term “butanol dehydrogenase” also refers to an enzyme that catalyzes the conversion of butyraldehyde to 1-butanol, using either NADH or NADPH as a cofactor. Butanol dehydrogenases are available from, for example, C. acetobutylicum (GenBank NOs: NP_149325, NC_001988; NP_349891, NC_003030, NP_349892, and NC_003030) and E. coli (GenBank NOs: NP_417-484 and NC_000913).

The term “branched-chain keto acid dehydrogenase” refers to an enzyme that catalyzes the conversion of α-ketoisovalerate to isobutyryl-CoA (isobutyryl-coenzyme A), typically using NAD+ as an electron acceptor. Example branched-chain keto acid dehydrogenases are known by the EC number 1.2.4.4. Such branched-chain keto acid dehydrogenases are comprised of four subunits and are available from a vast array of microorganisms, including, but not limited to, B. subtilis (GenBank Nos: CAB14336, Z99116; CAB14335, Z99116; CAB14334, Z99116; and CAB14337, Z99116) and Pseudomonas putida (GenBank Nos: AAA65614, M57613; AAA65615, M57613; AAA65617, M57613; and AAA65618, M57613).

The term “acylating aldehyde dehydrogenase” refers to an enzyme that catalyzes the conversion of isobutyryl-CoA to isobutyraldehyde, typically using either NADH or NADPH as an electron donor. Example acylating aldehyde dehydrogenases are known by the EC numbers 1.2.1.10 and 1.2.1.57. Such enzymes are available from multiple sources, including, but not limited to, Clostridium beijerinckii (GenBank Nos: AAD31841, AF157306), C. acetobutylicum (GenBank Nos: NP_149325, NC_001988; NP_149199, NC_001988), P. putida (GenBank Nos: AAA89106, U13232), and Thermus thermophilus (GenBank Nos: YP_145486, NC_006461).

The term “transaminase” refers to an enzyme that catalyzes the conversion of α-ketoisovalerate to L-valine, using either alanine (alanine-dependent) or glutamate (glutamate-dependent) as an amine donor. Example transaminases are known by the EC numbers 2.6.1.42 and 2.6.1.66. Such enzymes are available from a number of sources. Examples of sources of alanine-dependent enzymes include, but are not limited to, E. coli (GenBank Nos: YP_026231, NC_000913) and Bacillus lichenformis (GenBank Nos: YP_093743, NC_006322). Examples of sources of glutamate-dependent enzymes include, but are not limited to, E. coli (GenBank Nos: YP_026247, NC_000913), Saccharomyces cerevisiae (GenBank Nos: NP_012682, NC_001142) and Methanobacterium thermoautotrophicum (GenBank Nos: NP_276546, NC_000916).

The term “valine dehydrogenase” refers to an enzyme that catalyzes the conversion of α-ketoisovalerate to L-valine, typically using NAD(P)H as an electron donor and ammonia as an amine donor. Example valine dehydrogenases are known by the EC numbers 1.4.1.8 and 1.4.1.9 and such enzymes are available from a number of sources, including, but not limited to, Streptomyces coelicolor (GenBank Nos: NP_628270, NC_003888) and B. subtilis (GenBank Nos: CAB14339, Z99116).

The term “valine decarboxylase” refers to an enzyme that catalyzes the conversion of L-valine to isobutylamine and CO₂. Example valine decarboxylases are known by the EC number 4.1.1.14. Such enzymes are found in Streptomyces, such as Streptomyces viridifaciens (GenBank Nos: AAN10242, AY116644).

The term “omega transaminase” refers to an enzyme that catalyzes the conversion of isobutylamine to isobutyraldehyde using a suitable amino acid as an amine donor. Example omega transaminases are known by the EC number 2.6.1.18 and are available from a number of sources, including, but not limited to, Alcaligenes denitrificans (AAP92672, AY330220), Ralstonia eutropha (GenBank Nos: YP_294474, NC_007347), Shewanella oneidensis (GenBank Nos: NP_719046, NC_004347), and P. putida (GenBank Nos: AAN66223, AE016776).

The term “acetyl-CoA acetyltransferase” refers to an enzyme that catalyzes the conversion of two molecules of acetyl-CoA to acetoacetyl-CoA and coenzyme A (CoA). Example acetyl-CoA acetyltransferases are acetyl-CoA acetyltransferases with substrate preferences (reaction in the forward direction) for a short chain acyl-CoA and acetyl-CoA and are classified as E.C. 2.3.1.9 [Enzyme Nomenclature 1992, Academic Press, San Diego], although enzymes with a broader substrate range (E.C. 2.3.1.16) will be functional as well. Acetyl-CoA acetyltransferases are available from a number of sources, for example, E. coli (GenBank Nos: NP_416728, NC_000913; NCBI (National Center for Biotechnology Information) amino acid sequence, NCBI nucleotide sequence), C. acetobutylicum (GenBank Nos: NP_349476.1, NC_003030; NP_149242, NC_001988, B. subtilis (GenBank Nos: NP_390297, NC_000964), and S. cerevisiae (GenBank Nos: NP_015297, NC_001148).

The term “3-hydroxybutyryl-CoA dehydrogenase” refers to an enzyme that catalyzes the conversion of acetoacetyl-CoA to 3-hydroxybutyryl-CoA. 3-hydroxybutyryl-CoA dehydrogenases may be NADH-dependent, with a substrate preference for (S)-3-hydroxybutyryl-CoA or (R)-3-hydroxybutyryl-Co, and may be classified as E.C. 1.1.1.35 and E.C. 1.1.1.30, respectively. A 3-hydroxybutyryl-CoA dehydrogenase may be NADPH-dependent, having a substrate preference for (S)-3-hydroxybutyryl-CoA or (R)-3-hydroxybutyryl-CoA, and classified as E.C. 1.1.1.157 and E.C. 1.1.1.36, respectively. 3-Hydroxybutyryl-CoA dehydrogenases are available from a number of sources, for example, C. acetobutylicum (GenBank NOs: NP_349314, NC_003030), B. subtilis (GenBank NOs: AAB09614, U29084), Ralstonia eutropha (GenBank NOs: YP_294481, NC_007347), and Alcaligenes eutrophus (GenBank NOs: AAA21973, J04987).

The term “crotonase” refers to an enzyme that catalyzes the conversion of 3-hydroxybutyryl-CoA to crotonyl-CoA and H₂O. An example crotonase may have a substrate preference for (S)-3-hydroxybutyryl-CoA or (R)-3-hydroxybutyryl-CoA and may be classified as E.C. 4.2.1.17 and E.C. 4.2.1.55, respectively. Crotonases are available from a number of sources, for example, E. coli (GenBank NOs: NP_415911, NC_000913), C. acetobutylicum (GenBank NOs: NP_349318, NC_003030), B. subtilis (GenBankNOs: CAB13705, Z99113), and Aeromonas caviae (GenBank NOs: BAA21816, D88825).

The term “butyryl-CoA dehydrogenase” refers to an enzyme that catalyzes the conversion of crotonyl-CoA to butyryl-CoA. Example butyryl-CoA dehydrogenases may be NADH-dependent, NADPH-dependent, or flavin-dependent and may be classified as E.C. 1.3.1.44, E.C. 1.3.1.38, and E.C. 1.3.99.2, respectively. Butyryl-CoA dehydrogenases are available from a number of sources, for example, C. acetobutylicum (GenBank NOs: NP_347102, NC 003030), Euglena gracilis (GenBank NOs: Q5EU90, AY741582), Streptomyces collinus (GenBank NOs: AAA92890, U37135), and Streptomyces coelicolor (GenBank NOs: CAA22721, AL939127).

The term “butyraldehyde dehydrogenase” refers to an enzyme that catalyzes the conversion of butyryl-CoA to butyraldehyde, using NADH or NADPH as cofactor. Butyraldehyde dehydrogenases with preferences for NADH cofactors are known as E.C. 1.2.1.57 and are available from, for example, Clostridium beijerinckii (GenBank NOs: AAD31841, AF157306) and C. acetobutylicum (GenBank NOs: NP_149325, NC_001988).

The term “isobutyryl-CoA mutase” refers to an enzyme that catalyzes the conversion of butyryl-CoA to isobutyryl-CoA. Isobutyryl-CoA mutase may use coenzyme B12 as cofactor. Example isobutyryl-CoA mutases are known by the EC number 5.4.99.13. These enzymes are found in a number of Streptomyces, including, but not limited to, Streptomyces cinnamonensis (GenBank Nos: AAC08713, U67612; CAB59633, AJ246005), Streptomyces coelicolor (GenBank Nos: CAB70645, AL939123; CAB92663, AL939121), and Streptomyces avermitilis (GenBank Nos: NP_824008, NC_003155; NP_824637, NC_003155).

The term “acetolactate decarboxylase” refers to a polypeptide (or polypeptides) having an enzyme activity that catalyzes the conversion of alpha-acetolactate to acetoin. Example acetolactate decarboxylases are known as EC 4.1.1.5 and are available from, for example, B. subtilis (GenBank Nos: AAA22223, L04470), Klebsiella terrigena (GenBank Nos: AAA25054, L04507) and Klebsiella pneumoniae (GenBank Nos: AAU43774, AY722056).

The term “acetoin aminase” or “acetoin transaminase” refers to a polypeptide (or polypeptides) having an enzyme activity that catalyzes the conversion of acetoin to 3-amino-2-butanol. Acetoin aminase may use the cofactor pyridoxal 5′-phosphate, NADH, or NADPH. The resulting product may have (R) or (S) stereochemistry at the 3-position. The pyridoxal phosphate-dependent enzyme may use an amino acid, such as alanine or glutamate, as the amino donor. The NADH- and NADPH-dependent enzymes may use ammonia as a second substrate. A suitable example of an NADH-dependent acetoin aminase, also known as amino alcohol dehydrogenase, is described by Ito, et al. (U.S. Pat. No. 6,432,688). An example of a pyridoxal-dependent acetoin aminase is the amine:pyruvate aminotransferase (also called amine:pyruvate transaminase) described by Shin and Kim (J. Org. Chem. 67:2848-2853, 2002).

The term “acetoin kinase” refers to a polypeptide (or polypeptides) having an enzyme activity that catalyzes the conversion of acetoin to phosphoacetoin. Acetoin kinase may use ATP (adenosine triphosphate) or phosphoenolpyruvate as the phosphate donor in the reaction. Enzymes that catalyze the analogous reaction on the similar substrate dihydroxyacetone include, for example, enzymes known as EC 2.7.1.29 (Garcia-Alles, et al., Biochemistry 43:13037-13046, 2004).

The term “acetoin phosphate aminase” refers to a polypeptide (or polypeptides) having an enzyme activity that catalyzes the conversion of phosphoacetoin to 3-amino-2-butanol O-phosphate. Acetoin phosphate aminase may use the cofactor pyridoxal 5′-phosphate, NADH, or NADPH. The resulting product may have (R) or (S) stereochemistry at the 3-position. The pyridoxal phosphate-dependent enzyme may use an amino acid, such as alanine or glutamate, as the amino donor. The NADH- and NADPH-dependent enzymes may use ammonia as a second substrate. Although there are no reports of enzymes catalyzing this reaction on phosphoacetoin, there is a pyridoxal phosphate-dependent enzyme that is proposed to carry out the analogous reaction on the similar substrate serinol phosphate (Yasuta, et al., Appl. Environ. Microbial. 67:4999-5009, 2001).

The term “aminobutanol phosphate phospholyase,” also called “amino alcohol O-phosphate lyase,” refers to a polypeptide (or polypeptides) having an enzyme activity that catalyzes the conversion of 3-amino-2-butanol 0-phosphate to 2-butanone. Aminobutanol phosphate phospho-lyase may use the cofactor pyridoxal 5′-phosphate. There are reports of enzymes that catalyze the analogous reaction on the similar substrate 1-amino-2-propanol phosphate (Jones, et al., Biochem J 134:167-182, 1973). U.S. Pat. Pub. No. 2007/0259410 describes an aminobutanol phosphate phospholyase from the organism Erwinia carotovora.

The term “aminobutanol kinase” refers to a polypeptide (or polypeptides) having an enzyme activity that catalyzes the conversion of 3-amino-2-butanol to 3-amino-2-butanol 0-phosphate. Aminobutanol kinase may use ATP as the phosphate donor. Although there are no reports of enzymes catalyzing this reaction on 3-amino-2-butanol, there are reports of enzymes that catalyze the analogous reaction on ethanolamine and 1-amino-2-propanol (Jones, et al., Biochem J. 134:167-182, 1973). U.S. Pat. Pub. No. 2009/0155870 describes, in Example 14, an amino alcohol kinase of Erwinia carotovora subspecies atroseptica.

The term “butanediol dehydrogenase” also known as “acetoin reductase” refers to a polypeptide (or polypeptides) having an enzyme activity that catalyzes the conversion of acetoin to 2,3-butanediol. Butanedial dehydrogenases are a subset of the larger family of alcohol dehydrogenases. Butanediol dehydrogenase enzymes may have specificity for production of (R)- or (S)-stereochemistry in the alcohol product. (S)-specific butanediol dehydrogenases are known as EC 1.1.1.76 and are available from, for example, Klebsiella pneumoniae (GenBank Nos: BBA13085, D86412). (R)-specific butanediol dehydrogenases are known as EC 1.1.1.4 and are available from, for example, Bacillus cereus (GenBank Nos. NP_830481, NC_004722; AAP07682, AE017000), and Lactococcus lactis (GenBank Nos. AAK04995, AE006323).

The term “butanediol dehydratase,” also known as “dial dehydratase” or “propanediol dehydratase” refers to a polypeptide (or polypeptides) having an enzyme activity that catalyzes the conversion of 2,3-butanediol to 2-butanone. Butanediol dehydratase may use the cofactor adenosyl cobalamin. Adenosyl cobalamin-dependent enzymes are known as EC 4.2.1.28 and are available from, for example, Klebsiella oxytoca (GenBank Nos: AA08099 (alpha subunit), D45071; BAA08100 (beta subunit), D45071; and BBA08101 (gamma subunit), D45071 (all three subunits may be required for activity)), and Klebsiella pneumonia (GenBank Nos: AAC98384 (alpha subunit), AF102064; GenBank Nos: AAC98385 (beta subunit), AF102064, GenBank Nos: AAC98386 (gamma subunit), AF102064). Other suitable dial dehydratases include, but are not limited to, B12-dependent dial dehydratases available from Salmonella typhimurium (GenBank Nos: AAB84102 (large subunit), AF026270; GenBank Nos: AAB84103 (medium subunit), AF026270; GenBank Nos: AAB84104 (small subunit), AF026270); and Lactobacillus collinoides (GenBank Nos: CAC82541 (large subunit), AJ297723; GenBank Nos: CAC82542 (medium subunit); AJ297723; GenBank Nos: CAD01091 (small subunit), AJ297723); and enzymes from Lactobacillus brevis (particularly strains CNRZ 734 and CNRZ 735, Speranza, et al., J. Agric. Food Chem. 45:3476-3480, 1997), and nucleotide sequences that encode the corresponding enzymes. Methods of diol dehydratase gene isolation are well known in the art.

The term “pyruvate decarboxylase” refers to an enzyme that catalyzes the decarboxylation of pyruvic acid to acetaldehyde and CO₂. Pyruvate dehydrogenases are known by the EC number 4.1.1.1. These enzymes are found in a number of yeasts, including Saccharomyces cerevisiae (GenBank Nos: CAA97575, CAA97705, CAA97091).

Host cells comprising an isobutanol biosynthetic pathway as provided herein may further comprise one or more additional modifications. U.S. Pat. Pub. No. 2009/0305363, which is incorporated herein by reference in its entirety, discloses increased conversion of pyruvate to acetolactate by engineering yeast for expression of a cytosol-localized acetolactate synthase and substantial elimination of pyruvate decarboxylase activity. In some embodiments, the host cells comprise modifications to reduce glycerol-3-phosphate dehydrogenase activity, to disrupt at least one gene encoding a polypeptide having pyruvate decarboxylase activity, and/or to disrupt at least one gene encoding a regulatory element controlling pyruvate decarboxylase gene expression, as described in U.S. Pat. Pub. No. 2009/0305363. Modifications to a host cell that provide for increased carbon flux through an Entner-Doudoroff Pathway or reducing equivalents balance are described in U.S. Pat. Pub. No. 2010/0120105, which is incorporated herein by reference in its entirety. Other modifications include integration of at least one polynucleotide encoding a polypeptide that catalyzes a step in a pyruvate-using biosynthetic pathway.

Other modifications include at least one deletion, mutation, and/or substitution in an endogenous polynucleotide encoding a polypeptide having acetolactate reductase activity. As used herein, “acetolactate reductase activity” refers to the activity of any polypeptide having the ability to catalyze the conversion of acetolactate to 2,3-dihydroxy-2-methyl butyrate (DHMB). DHMB includes “fast DHMB,” which has the 2S, 3S configuration, and “slow DHMB,” which has the 2S, 3R configuration (See Kaneko et al., Phytochemistry 39: 115-120 (1995), which refers to “fast DHMB” as anglyceric acid and “slow DHMB” as triglyceric acid). In some embodiments, the polypeptide having acetolactate reductase activity is YMR226C of Saccharomyces cerevisiae or a homolog thereof.

Additional modifications include a deletion, mutation, and/or substitution in an endogenous polynucleotide encoding a polypeptide having aldehyde dehydrogenase and/or aldehyde oxidase activity, including a polypeptide that catalyzes the oxidation (dehydrogenation) of aldehydes, a polypeptide that catalyzes the conversion of isobutyraldehyde to isobutyric acid, and a polypeptide that corresponds to Enzyme Commission Numbers EC 1.2.1.3, EC 1.2.1.4 or EC 1.2.1.5. Aldehyde oxidases include a polypeptide that catalyzes production of carboxylic acids from aldehydes. Such polypeptides include a polypeptide that catalyzes the conversion of isobutyraldehyde to isobutyric acid and a polypeptide that corresponds to Enzyme Commission Number EC 1.2.3.1. In some embodiments, the polypeptide having aldehyde dehydrogenase activity is ALD6 from Saccharomyces cerevisiae or a homolog thereof.

A genetic modification that has the effect of reducing glucose repression in a PDC-yeast host cell is disclosed in U.S. Pat. Pub. No. 2011/0124060, incorporated herein by reference. The term “PDC-” refers to a cell that has a genetic modification to inactivate or reduce expression of at least one gene encoding pyruvate decarboxylase (PDC) such that the cell substantially or completely lacks pyruvate decarboxylase enzyme activity. If the yeast cell has more than one expressed (active) PDC gene, then each of the active PDC genes may be inactivated or have minimal expression, thereby producing a PDC− cell. In some embodiments, the pyruvate decarboxylase that is deleted or downregulated is selected from the group consisting of: PDC1, PDC5, PDC6, and combinations thereof. In some embodiments, the pyruvate decarboxylase is selected from PDC1 pyruvate decarboxylase from Saccharomyces cerevisiae, PDC5 pyruvate decarboxylase from Saccharomyces cerevisiae, PDC6 pyruvate decarboxylase from Saccharomyces cerevisiae, pyruvate decarboxylase from Candida glabrata, PDC1 pyruvate decarboxylase from Pichia stipites, PDC2 pyruvate decarboxylase from Pichia stipites, pyruvate decarboxylase from Kluveromyces lactis, pyruvate decarboxylase from Yarrowia lipolytica, pyruvate decarboxylase from Schizosaccharomyces pombe, and pyruvate decarboxylase from Zygosaccharomyces rouxii. In some embodiments, host cells contain a deletion or down-regulation of a polynucleotide encoding a polypeptide that catalyzes the conversion of glyceraldehyde-3-phosphate to glycerate 1,3, bisphosphate. In some embodiments, the enzyme that catalyzes this reaction is glyceraldehyde-3-phosphate dehydrogenase.

In an isobutanologen (PDC−) strain, PDC is deleted, the PDH pathway remains intact, and isobutanol production pathway enzymes are introduced. Often, the first enzyme to act in the isobutanol production pathway is acetolactate synthase (ALS). In isobutanologens, the carbon flux distribution for biomass growth and for the isobutanol pathway under aerobic conditions depends on the relative activity of ALS instead of the PDH enzyme. The physiological behavior of a recombinant isobutanologen is different from an unmodified S. cerevisiae due to the effect of the deletion of PDC genes and introduction of heterologous isobutanol pathway enzymes. To maximize biomass production in a recombinant isobutanologen in aerobic growth phase, the carbon flux has to channel through the PDH pathway efficiently to improve biomass yield and minimize carbon flux to isobutanol pathway leakages. Pathway leakage products may include isobutanol and isobutyric acid, which may adversely affect biomass growth rate and the final biomass achieved. In the production phase, the isobutanol yield and productivity may be adversely affected by accumulation of pathway intermediates (e.g., glycerol and isobutyric acid). Thus, the optimal operating regime (growth and production) for an ethanologen may not be the optimal operating regime for an isobutanologen.

International Pat. Pub. No. WO 2011/103300 discloses recombinant host cells comprising (a) at least one heterologous polynucleotide encoding a polypeptide having dihydroxyacid dehydratase activity; and (b)(i) at least one deletion, mutation, and/or substitution in an endogenous gene encoding a polypeptide affecting Fe—S cluster biosynthesis; and/or (b)(ii) at least one heterologous polynucleotide encoding a polypeptide affecting Fe—S cluster biosynthesis. In some embodiments, the polypeptide affecting Fe—S cluster biosynthesis is encoded by AFT1, AFT2, FRA2, GRX3, or CCC1. In some embodiments, the polypeptide affecting Fe—S cluster biosynthesis is constitutive mutant AFT1 L99A, AFT1 L102A, AFT1 C291F, or AFT1 C293F.

Additionally, host cells may comprise heterologous polynucleotides encoding a polypeptide with phosphoketolase activity and/or a heterologous polynucleotide encoding a polypeptide with phosphotransacetylase activity.

Also, given that accumulation of an alcohol during fermentation may be toxic to some microorganisms, as discussed above, recombinant microorganisms may be engineered to have a greater tolerance for alcohol. Examples of genetic modifications that may improve tolerance to alcohols include, but are not limited to, expression and/or modifications of relA, spoT, and dksA genes (described in U.S. Pat. Pub. No. 2009/0203139, incorporated herein by reference), elongase genes (Yazawa, et al., Appl. Microbiol. Biotechnol. 91:1593-1600, 2011), heat shock proteins (HSPs), as well as genes associated with lipid and fatty acid metabolism and cell membrane composition (see, e.g. Ma, et al., Appl. Microbiol. Biotechnol. 87:829-845, 2010). In addition, recombinant microorganisms may be engineered to have a certain level of thermotolerance, for example, by genetically modifying the microorganism to express stress-related genes, such as the genes encoding proteins involved in the ubiquitination process.

In some embodiments, any particular nucleic acid molecule or polypeptide may be at least 80%, at least 85%, at least 90%, at least 95%, at least 96%, at least 97%, at least 98%, or at least 99% identical to a nucleotide sequence or polypeptide sequence described herein. The term “percent identity,” as known in the art, is a relationship between two or more polypeptide sequences or two or more polynucleotide sequences, as determined by comparing the sequences. “Identity” may also refer to the degree of sequence relatedness between polypeptide or polynucleotide sequences, as determined by the match between strings of such sequences. “Identity” and “similarity” may be readily calculated by known methods, including but not limited to those disclosed in Computational Molecular Biology (Lesk, A. M., Ed.) Oxford University: NY (1988); Biocomputing: Informatics and Genome Projects (Smith, D. W., Ed.) Academic: NY (1993); Computer Analysis of Sequence Data, Part I (Griffin, A. M., and Griffin, H. G., Eds.) Humania: NJ (1994); Sequence Analysis in Molecular Biology (von Heinje, G., Ed.) Academic (1987); and Sequence Analysis Primer (Gribskov, M. and Devereux, J., Eds.) Stockton: NY (1991).

Standard recombinant DNA and molecular cloning techniques are well known in the art and are described by Sambrook, et al. (Sambrook, J., Fritsch, E. F. and Maniatis, T. (Molecular Cloning: A Laboratory Manual; Cold Spring Harbor Laboratory Press, Cold Spring Harbor, 1989, here in referred to as Maniatis) and by Ausubel, et al. (Ausubel, et al., Current Protocols in Molecular Biology, published by Greene Publishing Assoc. and Wiley-Interscience, 1987). Examples of methods to construct microorganisms that comprise a butanol biosynthetic pathway are disclosed, for example, in U.S. Pat. No. 7,851,188 and U.S. Pat. Pub. Nos. 2007/0092957; 2007/0259410; 2007/0292927; 2008/0182308; 2008/0274525; 2009/0155870; 2009/0305363; and 2009/0305370, each of which is herein incorporated by reference.

Growth and/or Fermentation Media and Carbon Sources

Recombinant microorganism cells disclosed herein are contacted with suitable carbon sources, typically in fermentation media. Suitable carbon sources may include monosaccharides, such as fructose or glucose; disaccharides; oligosaccharides, such as lactose, maltose, galactose, or sucrose; polysaccharides, such as starch or cellulose, ethanol, lactate, succinate, glycerol, carbon dioxide, methanol, glucose, dextrose, fructose, lactose, sucrose, xylose, arabinose, dextrose, cellulose, methane, or amino acids; or mixtures thereof. Suitable carbon sources may also include unpurified mixtures from renewable feedstocks, such as cheese whey permeate, cornsteep liquor, sugar beet molasses, and barley malt. The source of carbon used in the present disclosure may encompass a wide variety of carbon containing substrates and may be selected based on a number of factors, such as the type of microorganism used. The source of carbon may affect the activity of fermentation enzymes and may be selected to enhance the activity of fermentation enzymes. Without being bound by theory, it is believed that fermentation enzymes may become active or more active when cells are grown in the presence of dextrose.

In some embodiments, the carbon source is selected from glucose, fructose, sucrose, and mixtures thereof, including mixtures thereof with C5 sugars, such as xylose and/or arabinose, for yeast cells modified to use C5 sugars. Sucrose may be derived from renewable sugar sources, such as sugar cane, sugar beets, cassava, sweet sorghum, or mixtures thereof. Glucose and dextrose may be derived from renewable grain sources via saccharification of starch-based feedstocks, including grains, such as corn, wheat, rye, barley, oats, or mixtures thereof. In addition, fermentable sugars may be derived from renewable cellulosic or lignocellulosic biomass via processes of pretreatment and saccharification, as described, for example, in U.S. Pat. Pub. No. 2007/0031918.

Biomass, when used in reference to a carbon source, refers to any cellulosic or lignocellulosic material and includes materials comprising cellulose and, optionally, further comprising hemicellulose, lignin, starch, oligosaccharides, and/or monosaccharides. Biomass may also comprise additional components, such as protein and/or lipid. Biomass may be derived from a single source, or biomass may comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, and wood and forestry waste. Examples of biomass include, but are not limited to, corn grain, corn cobs, crop residues such as corn husks, corn stover grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, or mixtures thereof.

In addition to an appropriate carbon source, the growth and/or fermentation media may contain additional components, such as suitable minerals, salts, cofactors, buffers and other components known to those skilled in the art that are suitable for the growth of the cultures and promotion of a selected enzymatic pathway. Like the carbon source, these additional components may affect fermentation and alcohol production and may be selected to increase alcohol production. For example, the growth and/or fermentation media may include a select buffer(s) at a selected concentration(s) to maximize alcohol production.

Optionally, the fermentation media may contain ethanol. In some embodiments, when a recombinant microorganism having a butanol biosynthetic pathway is used as microorganism for butanol production, supplementation of the fermentation medium with a 2-carbon substrate (e.g., ethanol) may facilitate the survival and growth of the recombinant microorganism. Thus, in some embodiments, ethanol may be supplied to the fermentation medium.

Suitable growth and/or fermentation media, for whole cells (e.g., whole yeast cells), include common commercially prepared media such as Luria Bertani (LB) broth, Sabouraud Dextrose (SD) broth, or Yeast Medium (YM) broth or broth that includes yeast nitrogen base, ammonium sulfate, and dextrose (as the carbon/energy source) or YPD Medium, a blend of peptone, yeast extract, and dextrose in optimal proportions for growing most Saccharomyces cerevisiae strains. Other defined or synthetic growth and/or fermentation media may also be used. The use of agents known to modulate catabolite repression directly or indirectly, e.g., cyclic adenosine 2′,3′-monophosphate (cAMP), may also be incorporated into the growth and/or fermentation medium.

Growth and/or Fermentation Conditions

Typically, growth and/or fermentation of cells (e.g., whole yeast cells) occurs at a temperature in the range of about 20° C. to about 40° C., or about 25° C. to about 35° C., in an appropriate medium. In some embodiments, growth and/or fermentation of cells occurs at a temperature of about 20° C., about 22° C., about 25° C., about 28° C., about 30° C., about 32° C., about 35° C., about 37° C., or about 40° C. In some aspects, growth and/or fermentation of cells occurs at a temperature of about 20° C. to about 35° C., or about 20° C. to about 30° C., or about 25° C. to about 30° C. Certain cells are more thermo-tolerant and, in such cells, growth and/or fermentation may occur at higher temperatures, such as about 42° C., about 45° C., about 47° C., and even above about 50° C. (e.g., for short periods of time).

Suitable pH for the growth and/or fermentation of cells may range from a pH of about 3.0 to about 9.0, or about 3.0 to about 7.5. In some embodiments, the pH for the growth and/or fermentation of cells ranges from about 4.0 to about 9.0, or about 4.0 to about 8.0. In some aspects, a pH range of about 4.0 to about 7.0, or about 4.0 to about 6.0, or about 4.5 to about 5.5, is used for the fermentation of whole cells.

Fermentations may be performed under aerobic or anaerobic conditions. In some embodiments, anaerobic, microaerobic or a combination of conditions are used for fermentation.

### Industrial Batch and Continuous Fermentations

Butanol and other alcohols may be produced using a batch method of fermentation. A classical batch fermentation is a closed system where the composition of the medium is set at the beginning of the fermentation and not subject to artificial alterations during the fermentation. A variation of the standard batch system is the fed-batch system. Fed-batch fermentation processes are also suitable and comprise a typical batch system with the exception that the carbon source or carbon substrate is added in increments as the fermentation progresses. Fed-batch systems are useful when catabolite repression is likely to inhibit the metabolism of the cells and/or when it is desirable to have limited amounts of the carbon source or carbon substrate in the media. Batch and fed-batch fermentations are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992), herein incorporated by reference.

Butanol or other alcohol products may also be produced using continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density, where cells are primarily in log phase growth. Continuous fermentation allows for the modulation of one factor or any number of factors that affect cell growth or end product concentration. Methods of modulating nutrients and growth factors for continuous fermentation processes as well as techniques for maximizing the rate of product formation are well known in the art of industrial microbiology.

It is contemplated that the production of butanol or other alcohol products may be practiced using batch, fed-batch, and/or continuous processes and that any known mode of fermentation may be suitable. Additionally, it is contemplated that cells may be immobilized on a substrate as whole cell catalysts and subjected to fermentation conditions for butanol production.

### Methods for Recovering Alcohol Using Extractive Fermentation

The renewable alcohol, e.g., butanol, may be recovered from a fermentation medium containing the alcohol, water, at least one fermentable carbon source, and a recombinant microorganism that has been genetically modified (genetically engineered) to produce the renewable alcohol via a biosynthetic pathway from the at least one carbon source. The first step in the process is contacting the fermentation medium with a water immiscible organic extractant to form an aqueous phase and a non-aqueous organic extractant phase (or a biphasic/two-phase mixture comprising an aqueous phase and a non-aqueous organic extractant phase). The water immiscible organic extractant or water immiscible organic extractant composition may comprise diesel fuel. In addition to diesel fuel, the water immiscible organic extractant composition may optionally comprise one or more solvents (e.g., organic solvent).

Suitable diesel fuels include, for example, fossil diesel, petroleum diesel, synthetic diesel, biodiesel, renewable diesel, hydrogenated oils and fats, and the like, or combinations thereof. Petroleum diesel or fossil diesel is produced from the fractional distillation of crude oil at a temperature between 200° C. and 350° C. and at atmospheric pressure, resulting in a mixture of carbon chains that typically contain between about 9 and about 25 carbon atoms per molecule. Synthetic diesel is produced from any carbonaceous material, including biomass, biogas, natural gas, coal and others. The raw material is gasified into synthesis gas, which, after purification, is converted by the Fischer-Tropsch process to a synthetic fuel. The process is typically referred to as biomass-to-liquid (BTL), gas-to-liquid (GTL) or coal-to-liquid (CTL), depending on the raw material used. Paraffinic synthetic diesel generally has a near-zero content of sulfur and very low aromatics content, reducing unregulated emissions of toxic hydrocarbons, nitrous oxides, and particulate matter (PM). E-diesel is a synthetic diesel fuel created from carbon dioxide, water, and electricity with a process powered by renewable energy sources to create a liquid energy carrier that is refined to generate e-diesel.

Biodiesel is obtained from vegetable oil or animal fats (biolipids), which are mainly fatty acid methyl esters (FAME), and transesterified with methanol. Biodiesel is produced from vegetable oils or animal fats by transesterification, which is a process that converts fats and oils into biodiesel and glycerin (a coproduct). Biodiesel may be produced from many types of oils, the most common being rapeseed oil (rapeseed methyl ester, RME) in Europe and soybean oil (soy methyl ester, SME) in the U.S. Methanol may also be replaced with ethanol for the transesterification process, which results in the production of ethyl esters. The transesterification processes use catalysts, such as sodium or potassium hydroxide, to convert vegetable oil and methanol into biodiesel and the byproducts glycerine and water, which are typically removed from the fuel along with traces of methanol. Pure biodiesel (e.g., B100) may be used in some engines, where the manufacturer approves such use. It is more common, though, for biodiesel to be blended with another fuel, such as petroleum diesel, and used as a mixture. Examples of biodiesel fuel blends include B2 (2% biodiesel, 98% petroleum diesel), B5 (up to 5% biodiesel, at least 95% petroleum diesel), B7 (up to 7% biodiesel, at least 93% petroleum diesel), B20 (6% to 20% biodiesel and 80% to 94% petroleum diesel), and B99 biodiesel (99.9% biodiesel and 0.1% petroleum diesel).

Renewable diesel (sometimes referred to as hydrotreated vegetable oil (HVO) or green diesel) may be produced from nearly any biomass feedstock, including those used for biodiesel production, through a variety of processes, such as hydrotreating, gasification, pyrolysis, or other biochemical and thermochemical technologies. Renewable diesel is similar to biodiesel but with important differences. Renewable diesel is a hydrocarbon that is chemically equivalent to petroleum diesel. Therefore, renewable diesel may be used as a drop-in biofuel, transported in petroleum pipelines, and/or sold at retail stations with or without blending with petroleum diesel. Renewable diesel production uses a hydrogenation process rather than the esterification process used to produce biodiesel. Because renewable diesel is a drop-in fuel, it meets ASTM D975 specification for petroleum diesel and may be seamlessly blended, transported, and even co-processed with petroleum diesel.

Biodiesel and/or renewable diesel may be derived from biological sources, such as vegetables, animals, fish, and/or algae. Vegetable fats/oils refer generally to any plant-based material and may include fat/oils derived from a plant source, such as plants of the genus Jatropha. Suitable biological sources include vegetable fats/oils, animal fats/oils, fish oils, pyrolysis oils, and algae lipids/oils, as well as one or more types of lipids or lipid compounds. Major classes of lipids include, but are not necessarily limited to, fatty acids, glycerol-derived lipids (including fats, oils and phospholipids), sphingosine-derived lipids (including ceramides, cerebrosides, gangliosides, and sphingomyelins), steroids and their derivatives, terpenes and their derivatives, fat-soluble vitamins, certain aromatic compounds, and long-chain alcohols and waxes. In living organisms, lipids generally serve as the basis for cell membranes and as a form of fuel storage. Lipids may also be found conjugated with proteins or carbohydrates, such as in the form of lipoproteins and lipopolysaccharides.

Vegetable oils that may be used in accordance with this disclosure include, but are not limited to rapeseed (canola) oil, soybean oil, coconut oil, sunflower oil, palm oil, palm kernel oil, peanut oil, linseed oil, tall oil, corn oil, castor oil, jatropha oil, jojoba oil, olive oil, flaxseed oil, camelina oil, safflower oil, babassu oil, tallow oil, and rice bran oil. As used herein, vegetable oils also include processed vegetable oils or vegetable oil material. Non-limiting examples of processed vegetable oil material include fatty acids and fatty acid alkyl esters, such as C1-C5 fatty acid alkyl esters (e.g., fatty acid methyl esters, fatty acid ethyl esters, fatty acid propyl esters).

Animal fats that may be used in accordance with the disclosure include, but are not limited to, beef fat (tallow), hog fat (lard), turkey fat, fish fat/oil, and chicken fat. The animal fats may be obtained from any suitable source including restaurants and meat production facilities. As used herein, animal fats also include processed animal fats or animal fat material. Non-limiting examples of processed animal fat material include fatty acids and fatty acid alkyl esters, such as C1-C5 fatty acid alkyl esters (e.g., fatty acid methyl esters, fatty acid ethyl esters, fatty acid propyl esters).

Algae oils or lipids are typically contained in algae in the form of membrane components, storage products, and metabolites. Certain algal strains, particularly microalgae such as diatoms and cyanobacteria, contain proportionally high levels of lipids. Algal sources for algae oils or lipids may contain varying amounts, e.g., from about 2 wt. % to about 40 wt. % of oils or lipids, based on a total weight of the biomass itself. Algal sources for algae oils include, but are not limited to, unicellular and multicellular algae. Examples of such algae include rhodophyta, chlorophyta, heterokontophyta, tribophyta, glaucophyta, chlorarachniophyta, euglenoid, haptophyta, cryptomonads, dinoflagellates, phytoplankton, and the like, and combinations thereof. In some examples, algae may be of the classes Chlorophyceae and/or Haptophyta. Specific species may include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlanmydomonas reinhardtii.

Suitable biodiesel fuels include any of those that comprise primarily triglycerides and free fatty acids (FFAs). The triglycerides and FFAs typically contain aliphatic hydrocarbon chains having from 8 to 36 carbons, or from 10 to 26 carbons, or from 14 to 22 carbons. Types of triglycerides may be determined according to their fatty acid constituents (it is understood that the fatty acid constituent or component need not necessarily contain a carboxylic acid hydrogen). A fat or oil may be analyzed to determine its fatty acid constituents using, for example, gas chromatography (GC). This analysis may involve extracting the fat or oil, saponifying (hydrolyzing) the fat or oil, preparing an alkyl (e.g., methyl) ester of the saponified fat or oil, and determining the type of (methyl) ester using GC analysis. In some instances, a majority or greater than 50% of the triglycerides present in a fat, oil, or lipid material, based on total triglyceride present in the lipid material, contains C10 to C26, or C12 to C24, fatty acid constituents. Other types of feed that are derived from biological raw material components may include fatty acid esters, such as fatty acid alkyl esters (e.g., FAME and/or fatty acid ethyl ester (FAEE)).

Biodiesel fuels typically have relatively low nitrogen and sulfur contents. For example, a biodiesel fuel may contain up to about 500 wppm nitrogen, or up to about 300 wppm nitrogen or up to about 100 wppm nitrogen. Instead of nitrogen and/or sulfur, the primary heteroatom component in biodiesel fuels is oxygen. Biodiesel fuels may include up to about 10 wt. % oxygen, or up to about 12 wt. % oxygen, or up to about 14 wt. % oxygen. Prior to optional hydrotreatment, suitable biodiesel fuels may include at least about 5 wt. % oxygen, or at least about 8 wt. % oxygen.

In some instances, the diesel fuel is derived from up to about 100% of a feedstock having a biological origin or the diesel fuel comprises up to about 100% of a fuel having a biological origin. The feedstock may optionally be hydrotreated, such as a hydrotreated vegetable oil feed, hydrotreated fatty acid alkyl ester feed, or another type of hydrotreated biological feed, to reduce the oxygen content of the feed to about 500 wppm or less, or about 200 wppm or less, or about 100 wppm or less. Additionally or alternately, a biological feed may be blended with a mineral feed, so that the blended feed may be tailored to have an oxygen content of about 500 wppm or less, or about 200 wppm or less, or about 100 wppm or less. In some instances, at least a portion of the feedstock is of a biological origin, for example, at least about 2 wt. %, or at least about 5 wt. %, or at least about 10 wt. %, or at least about 20 wt. %, or at least about 25 wt. %, or at least about 35 wt. %, or at least about 50 wt. %, or at least about 60 wt. %, or at least about 75 wt. %. In certain instances, the biological portion may be about 75 wt. % or less, or about 60 wt. % or less, or about 50 wt. % or less, or about 35 wt. % or less, or about 25 wt. % or less, or about 20 wt. % or less, or about 10 wt. % or less, or about 5 wt. % or less.

As discussed above, in addition to diesel fuel, the water immiscible organic extractant composition may optionally comprise one or more solvents (e.g., organic solvents). Suitable solvents include C4 to C22 fatty alcohols, C4 to C28 fatty acids, esters of C4 to C28 fatty acids, C4 to C22 fatty aldehydes, C7 to C22 ethers, amides, phosphate esters, ureas, phenols (phenolics), phosphinates, carbamates, phosphoramide, or mixtures thereof. The solvent may be selected from the group consisting of oleyl alcohol, phenyl alcohol, cetyl alcohol, lauryl alcohol, myristyl alcohol, stearyl alcohol, oleic acid, lauric acid, myristic acid, stearic acid, octanoic acid, decanoic acid, undecanoic acid, methyl myristate, methyl oleate, 1-nonanol, 1-decanol, 2-undecanol, 1-nonanal, 1-undecanol, undecanal, isododecanol, lauric aldehyde, 2-methylundecanal, oleamide, linoleamide, palmitamide, stearylamide, 2-ethyl-1-hexanol, 2-hexyl-1-decanol, 2-octyl-1-dodecanol, octanol (e.g., 1-octanol), heptanol, phenetole, and mixtures thereof. The solvent may be polar and/or may include one or more of a phosphorous atom, a nitrogen atom, a sulfur atom, or an oxygen atom. The solvent may exhibit hydrogen bonding.

In some embodiments, the fermentation medium may contain ethanol. In some instances, the non-aqueous organic extractant composition or non-aqueous organic extractant phase may contain ethanol, in addition to diesel fuel. In some embodiments, where the non-aqueous organic extractant composition comprises one or more solvents, in addition to diesel fuel, the one or more solvents and the diesel fuel may first be combined together before contacting the fermentation medium. For example, diesel fuel and a first solvent may be combined in a vessel, such as a mixing tank, to form the extractant composition, which may then be added to a vessel containing the fermentation medium (e.g., fermentation vessel). Alternatively, diesel fuel and a first solvent need not be combined before contacting the fermentation medium and may instead be combined at the same time while contacting the fermentation medium. For example, diesel fuel and a first solvent may be separately added, simultaneously or at different times, to a vessel that contains the fermentation medium. In some embodiments, contacting the fermentation medium with the non-aqueous organic extractant composition comprises contacting the fermentation medium with diesel fuel and then contacting the fermentation medium and the diesel fuel with a first solvent. In some embodiments, the contacting with a first solvent occurs in the same vessel as the contacting with diesel fuel. In some embodiments, the contacting with a first solvent occurs in a different vessel than the contacting with diesel fuel. For example, the diesel fuel may be contacted with the fermentation medium in a first vessel, and the contents of the first vessel may then be transferred to a second vessel, in which contact with the first solvent occurs.

The non-aqueous organic extractant composition may contact the fermentation medium at the start of the fermentation to form a biphasic fermentation medium. Alternatively, the non-aqueous organic extractant composition may contact the fermentation medium after the microorganism has achieved a desired level of growth, which may be determined by measuring the optical density of the culture. Further, the non-aqueous organic extractant composition may contact the fermentation medium at a time at which the alcohol, e.g., butanol, level in the fermentation medium reaches a preselected level, for example, before the alcohol concentration reaches a level that is toxic to the fermentative microorganism. The alcohol, e.g., butanol, concentration may be monitored during the fermentation using methods known in the art, such as by gas chromatography or high-performance liquid chromatography.

Fermentation may be run under aerobic conditions for a time sufficient for the culture to achieve a preselected level of growth, as determined by optical density measurement. An inducer may optionally be added to induce the expression of the alcohol biosynthetic pathway in the recombinant or modified microorganism, and fermentation conditions may be switched to microaerobic or anaerobic conditions to stimulate butanol production, as described in detail in Example 6 of U.S. Pat. Pub. No. 2009/0305370 A1, where the extractant is added after the switch to microaerobic or anaerobic conditions.

Through contacting the fermentation medium with the non-aqueous organic extractant, the alcohol, e.g., butanol, partitions into the non-aqueous organic extractant phase, decreasing the concentration in the aqueous phase containing the microorganisms, thereby limiting the exposure of the microorganisms to the potentially toxic alcohol. The volume of the non-aqueous organic extractant to be used depends on a number of factors, including the volume of the fermentation medium, the size of the fermenter, the partition coefficient of the extractant for the alcohol, and the fermentation mode chosen, as described below. The volume of the non-aqueous organic extractant may be about 3% to about 60% of the fermenter working volume. The ratio of the non-aqueous organic extractant to the fermentation medium may be from about 1:20 to about 20:1 on a volume:volume basis, or from about 1:15 to about 15:1, or from about 1:12 to about 12:1, or from about 1:10 to about 10:1, or from about 1:9 to about 9:1, or from about 1:8 to about 8:1 on a volume:volume basis.

The next step may be separating the non-aqueous organic extractant phase, the alcohol-containing phase, from the aqueous phase using methods known in the art, including but not limited to, siphoning, decantation, centrifugation, using a gravity settler, membrane-assisted phase splitting, and the like. The non-aqueous organic extractant phase may be separated and recovered as a renewable fuel product—an isobutanol-diesel fuel blend. If an isobutanol-diesel fuel blend product is desired, fermentation may continue until a desired concentration of isobutanol is partitioned. Once the desired concentration of isobutanol is partitioned, the final isobutanol-diesel fuel blend product may be harvested and/or stored. Alternatively, the alcohol, e.g., isobutanol, may be recovered from the non-aqueous organic extractant (alcohol-containing) phase.

Recovery of the alcohol, e.g., butanol, from the non-aqueous organic extractant (alcohol-containing) phase may be done using methods known in the art, including but not limited to, distillation, adsorption by resins, separation by molecular sieves, pervaporation, and the like. Specifically, distillation may be used to recover the alcohol, e.g., butanol, from the non-aqueous organic extractant (alcohol-containing) phase. The non-aqueous organic extractant may be recycled to the alcohol, e.g., butanol, production and/or recovery process.

Gas stripping may be used concurrently with the non-aqueous organic extractant composition to remove the alcohol, e.g., butanol, from the fermentation medium. Gas stripping may be done by passing a gas such as air, nitrogen, or carbon dioxide through the fermentation medium, thereby forming an alcohol-containing gas phase. The alcohol product, e.g., butanol, may be recovered from the alcohol-containing gas phase using methods known in the art, such as using a chilled water trap to condense the alcohol or scrubbing the gas phase with a solvent.

Any alcohol, e.g., butanol, remaining in the fermentation medium after the fermentation run is completed may be recovered by continued extraction using fresh or recycled organic extractant. Alternatively, the alcohol product, e.g., butanol, may be recovered from the fermentation medium using methods known in the art, including, but not limited to distillation, azeotropic distillation, liquid-liquid extraction, adsorption, gas stripping, membrane evaporation, pervaporation, and the like.

The two-phase extractive fermentation method may be carried out in a continuous mode. In this mode, the mixture of the fermentation medium and the alcohol-containing organic extractant composition is removed. The two phases are separated by means known in the art including, but not limited to, siphoning, decantation, centrifugation, using a gravity settler, membrane-assisted phase splitting, and the like, as described above. After separation, the fermentation medium may be recycled or may be replaced with fresh medium. Then, the extractant may be treated to recover the alcohol product, e.g., butanol, as described above. The extractant may then be recycled for further extraction of the product. Alternatively, fresh extractant may be continuously added to replace the removed extractant. This continuous mode of operation may offer some advantages. Because the product is continually removed from the reactor, a smaller volume of organic extractant composition may be required, which may enable a larger volume of the fermentation medium to be used, resulting in higher production yields. The volume of the organic extractant composition may be about 3% to about 50% of the working volume, or about 3% to about 20% of the working volume; or about 3% to about 10% of the working volume. It may be beneficial to use the smallest concentration of extractant possible to maximize the volume of the aqueous phase, and therefore, the amount of fermentative microorganisms. The process may be operated in an entirely continuous mode, in which the extractant is continuously recycled. In this entirely continuous mode, the alcohol product, e.g., butanol, is not allowed to reach the critical toxic concentration. The apparatuses that may be used to carry out these modes of two-phase extractive fermentations are well known in the art. Examples are described in, for example, Kollerup et al. in U.S. Pat. No. 4,865,973.

A batch fermentation mode may also be used. Batch fermentation, which is well known in the art, is a closed system in which the composition of the fermentation medium is set at the beginning of the fermentation and is not subjected to artificial alterations during the process. In this mode, a volume of organic extractant composition is added to the fermenter, and the extractant is not removed during the process. The organic extractant composition may comprise diesel fuel and, optionally, one or more solvents, and the extractant composition may be formed in the fermenter by separate addition of diesel fuel and a first solvent. Alternatively, the organic extractant composition may comprise diesel fuel and one or more solvents, where the diesel fuel and a first solvent may be combined to form the extractant composition prior to the addition of the extractant composition to the fermenter. This mode may require a larger volume of organic extractant composition to minimize the concentration of the inhibitory alcohol product, e.g., butanol, in the fermentation medium.

Fed-batch fermentation mode may also be used. Fed-batch fermentation is a variation of the standard batch system, in which the nutrients, for example glucose, are added in increments during the fermentation. The amount and the rate of addition of the nutrient may be determined by routine experimentation. For example, the concentration of critical nutrients in the fermentation medium may be monitored during the fermentation. Alternatively, more easily measured factors such as pH, dissolved oxygen, and the partial pressure of waste gases, such as carbon dioxide, may be monitored. From these measured parameters, the rate of nutrient addition may be determined. The amount of organic extractant composition used and its methods of addition in this mode may be the same as in the batchwise mode, described above.

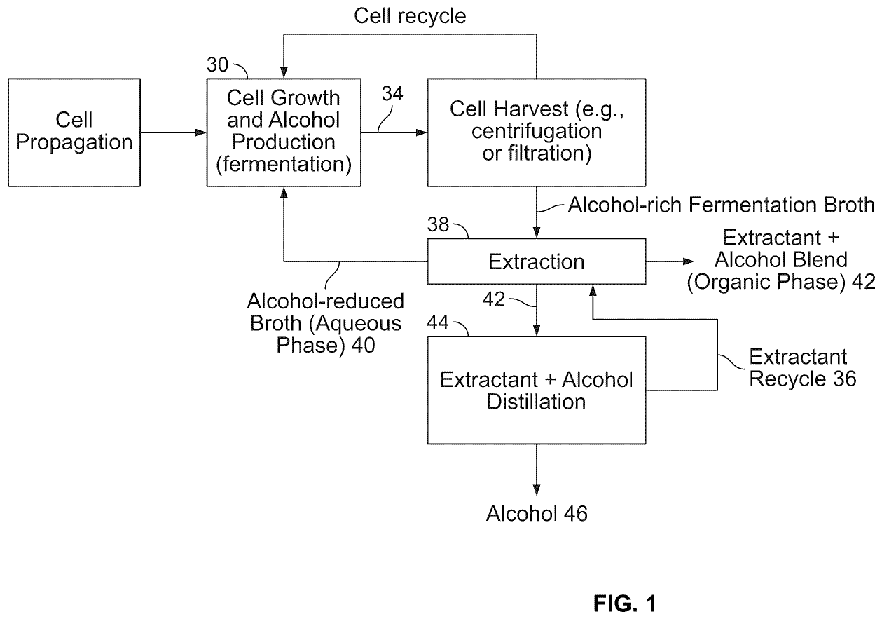

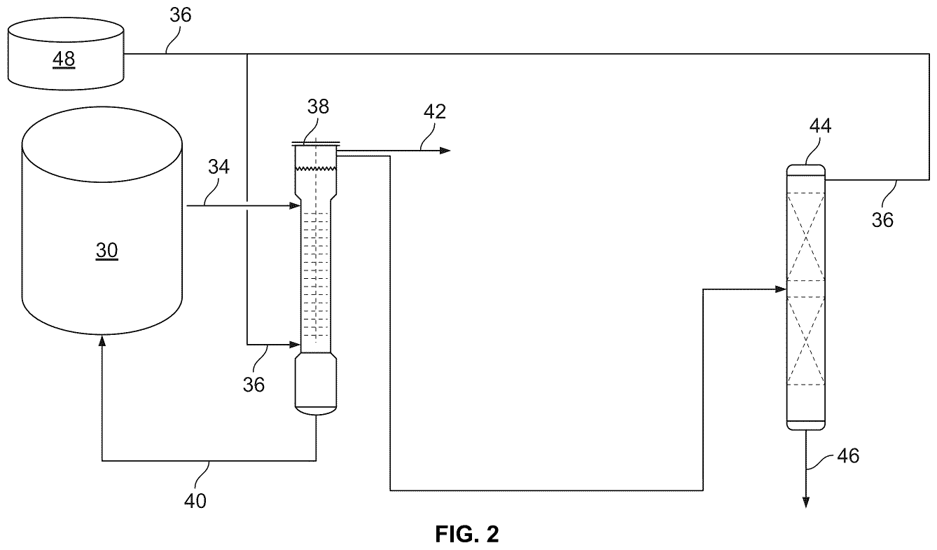

As shown in FIGS. 1 and 2, extraction of the alcohol, e.g., butanol, may occur downstream of the fermenter, rather than in situ, such that the extraction of the alcohol, e.g., butanol, into the organic extractant composition is carried out on the fermentation medium removed from the fermenter. The amount of organic extractant composition used is about 20% to about 60% of the working volume or 30% to about 60% of the working volume. In some instances, as shown in FIGS. 1 and 2, an organic extractant composition comprising diesel fuel 36 may be contacted with a fermentation broth 34 in a vessel or unit 38 that is separate from and, optionally, downstream of the fermentation vessel 30. Thus, extraction may be done downstream of the fermenter, rather than in situ. In some embodiments, the separate unit may be a liquid-liquid extraction unit 38, such as a column (e.g., counter-current extraction column). The extraction of the alcohol, e.g., butanol, by the organic extractant composition may be done with or without the removal of the recombinant microorganisms from the fermentation medium or broth. The recombinant microorganisms may be removed from the fermentation medium by means known in the art including, but not limited to, filtration or centrifugation. In some instances, as shown in FIG. 1, the microorganism(s) are removed from the fermentation broth to yield an alcohol-rich fermentation broth, and the alcohol-rich fermentation broth is contacted with the extractant composition to extract or partition the alcohol (e.g., butanol) from the fermentation broth, forming a non-aqueous organic phase (comprising a blend of extractant and alcohol) and an aqueous phase (comprising an alcohol-reduced broth). The microorganism(s) that are removed from the fermentation broth may be recycled to the fermentation vessel.

In some instances, a stream of the extractant composition 36 may be injected into the bottom of the liquid-liquid extraction unit 38, and a stream of the fermentation broth 34 may be injected into the top of the liquid-liquid extraction unit 38. The stream of the fermentation broth 34 may be fed directly or indirectly from the fermentation vessel 30 to the liquid-liquid extraction unit 38. The stream of the extractant composition 36 may be fed directly or indirectly from a source of extractant composition 48 to the liquid-liquid extraction unit 38. The injection of the stream of extractant composition 36 and the injection of the stream of fermentation broth 34 may occur simultaneously. Generally, the fermentation broth 34 is denser and flows down through the liquid-liquid extraction unit 38. In some instances, the extractant composition comprising diesel fuel 36 contacts the fermentation broth 34 and extracts alcohol (e.g., butanol) from the fermentation broth, forming a non-aqueous organic (alcohol-containing) extractant phase. An aqueous phase containing the fermentative microorganism (e.g., yeast), minus the alcohol, is also formed.

A stream of the aqueous phase containing the fermentative microorganism 40 may be recycled (e.g., pumped back) to the fermenter 30, as shown in FIG. 2, or discarded and replaced with fresh fermentation medium or broth. Alternatively, the stream of the aqueous phase containing the fermentative microorganism 40 may be further treated for the removal of any remaining alcohol, e.g., butanol, and the resulting isolated recombinant microorganisms may also be recycled to the fermenter 30 (not shown). The stream of the aqueous phase containing the fermentative microorganism 40 may be continuously or periodically removed from the liquid-liquid extraction unit 38. The non-aqueous organic extractant phase, which contains the diesel fuel and the alcohol, e.g., butanol, may be removed from the top of the column as an alcohol-diesel fuel blend product 42. Alternatively, the alcohol (e.g., butanol) may be separated and recovered from the non-aqueous organic extractant phase. In some instances, the non-aqueous organic extractant phase is directed to a distillation system 44 to remove the alcohol 46 as a product. After removal of the alcohol product, e.g., butanol, the organic extractant composition comprising the diesel fuel may optionally be recycled to be used again in the extraction process. For example, the extractant composition comprising the diesel fuel may optionally be recycled to the liquid-liquid extraction unit to be reused as an extractant 36.

### Further Processing of Renewable Alcohol

Renewable alcohols, such as ethanol and isobutanol, may be sold as commodity chemicals directly. Alternatively, renewable alcohols may be further processed. For example, the renewable alcohols may be dehydrated to their respective olefins (e.g. ethylene and isobutene and one or more renewable linear butenes-typically a mixture of isobutene, 1-butene and cis/trans-2-butene). The renewable ethylene and renewable butenes may then also either be sold directly or still further processed (e.g., separated or reacted) in a variety of different ways to produce a wide variety of renewable hydrocarbon product streams. In some embodiments, further processing may comprise mixing the renewable ethylene and/or butene with ethylene and/or butylene produced by conventional methods (e.g., petroleum cracking) to produce an array of hydrocarbon compounds comprising renewable carbon. Accordingly, such compounds, while not composed solely of renewable carbon, still comprise at least some renewable carbon, with concomitant environmental advantages.

In some embodiments, renewable butene may be produced via the dehydration of renewable isobutanol. The renewable butene formed is thereby typically a tunable mixture of butene isomers, which is easily separated from the isobutanol feed to the dehydration reaction and may be sold directly as a mixture or reacted as a mixture to form other hydrocarbons (e.g., polybutenes), or the mixture of renewable butene isomers may be separated (e.g., by distillation, by selective conversion, etc.) into individual butene isomers, which may then either be sold individually as feedstocks, polymerized (e.g. to renewable polyisobutylene or butene copolymers), oligomerized (e.g., dimerized, trimerized, etc.) to form higher molecular weight olefins (e.g. isooctene or pentamethylheptenes), isomerized (e.g. isobutene isomerized to linear butenes, 1-butene isomerized to 2-butene, or 2-butene isomerized to 1-butene, etc.), dehydrogenated (e.g. to butadiene), or undergo combinations of these processes. In particular, isobutene dimers and trimers may be hydrogenated to provide, e.g., renewable isooctane, which may be useful as a renewable transportation fuel or a renewable additive for transportation fuels.

In some embodiments, renewable ethylene may be produced via the dehydration of renewable ethanol. The renewable ethylene produced thereby is of very high purity and is easily separated from the unreacted feedstock of the dehydration reaction. The renewable ethylene may then be either sold directly as a feedstock, or subsequently converted to higher value renewable hydrocarbons, such as higher molecular weight olefins produced by oligomerization reactions (e.g. dimers, trimers, etc.), polymerized to form renewable polyethylene, oxidized to form renewable ethylene oxide (which may subsequently be polymerized to form renewable polyethylene oxide, or converted to other renewable polyethylene oxide derivatives), converted to dichloroethane (for subsequent conversion to vinyl chloride and polymerization thereof), or used as a renewable feedstock for alkylating other olefins or aromatics (e.g., alkylation of benzene to produce ethylbenzene).

### EXAMPLES

The following non-limiting examples will further illustrate the systems, methods, and compositions disclosed herein. It should be understood that, while the following examples involve glucose as the carbon source, other carbon sources, feedstocks, or biomass sources, such as corn, may be used for feedstock without departing from the present invention. Moreover, while the following examples involve butanol, other alcohols may be produced without departing from the present invention. It should also be understood that the experimental test methods described below, including the concentrations specified in the experimental test methods, are adapted for bench- or lab-scale production. The experimental test methods described below may be scaled up for commercial/industrial and/or pilot production.

### Example 1. Effect of Different Organic Extractants on the Isobutanol Yield of Batch Fermentation

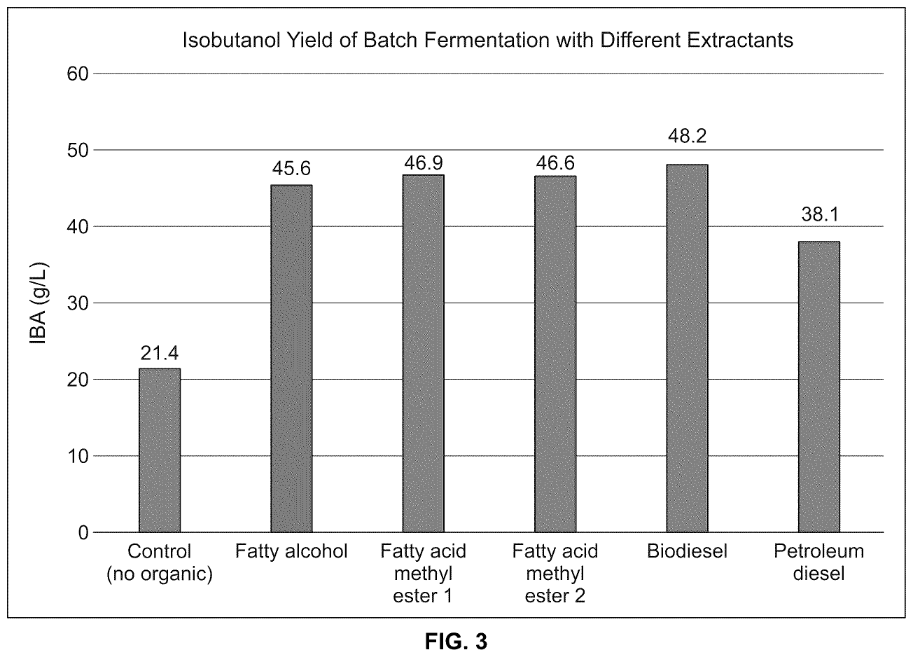

A Saccharomyces cerevisiae strain, which has deletions in pyruvate decarboxylase genes to restrict the conversion of pyruvate to EtOH and is engineered to produce isobutanol from a carbohydrate source, is propagated in a nutrient-rich media containing a non-fermentable carbon source to promote growth. After 24 hours of incubation, the cell culture is inoculated into 125 mL Erlenmeyer flasks containing 37 mL of nutrient media comprised of yeast extract and peptone. Glucose is added as the fermentable carbon source at a concentration of 150 g/L. After inoculation, 37 mL of organic extractant is overlayed onto the culture media and placed in a shaker/incubator. An extractant-free control is also included in the evaluation. Periodic sampling of the aqueous (containing the yeast culture) and organic (extractant) phases is performed, and samples are collected for analysis by gas chromatography. The graph of FIG. 3 shows that increased isobutanol titers are achieved after 48 hours of production, with the addition of fatty alcohols, fatty acid methyl esters, biodiesel, and petroleum diesel, as compared to a control sample that contains no organic extractant. In the control sample, the highest isobutanol titer that the yeast may tolerate under the conditions tested is 21.4 g/L. With the addition of fatty alcohols, fatty acid methyl esters, biodiesel, or petroleum diesel, the yeast remains viable and active, and removal of the isobutanol from the aqueous culture allows for continued production that yields from 38 g/L to 48 g/L of isobutanol.

### Example 2. Extraction of Isobutanol from an Active Fermentation into Biodiesel and Recovering the Isobutanol and Reusing the Biodiesel in Multiple Batches

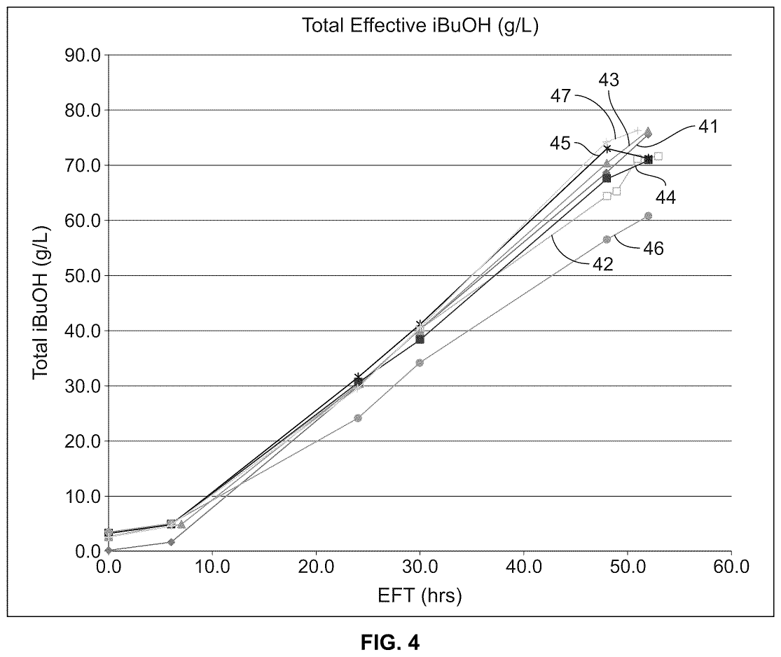

A Saccharomyces cerevisiae strain, which has deletions in pyruvate decarboxylase genes to restrict the conversion of pyruvate to EtOH and is engineered to produce isobutanol from a carbohydrate source, is propagated in a nutrient rich media containing a non-fermentable carbon source to promote cell growth. After 24 hours of incubation, the cell culture is inoculated into 2 L stirred tank fermenters containing corn mash and other nutrient supplements. Glucoamylase is added to hydrolyze the soluble oligomeric sugars to glucose. The yeast begins to consume glucose producing IBA under controlled conditions. During production, a continuous flow of fermentation culture is drawn from the fermentor and fed into the top of a liquid-liquid extraction (LLE) column. Simultaneously, biodiesel is fed into the bottom of the LLE column. The two streams interact in a counter current flow where the aqueous fermentor broth drops to the bottom and the organic solvent (biodiesel) flows to the top. The aqueous broth is returned to the fermentor to continue fermenting, and the organic solvent is removed from the top of the column and captured in a separate solvent tank, as displayed in FIG. 2. The fermentation and extraction continue until the glucose is consumed (48-52 hours). After completion of the batch, the organic solvent (biodiesel) is removed from the aqueous phase and distilled using a thin film evaporator. The IBA distillate is recovered, and the regenerated biodiesel is used in the next batch fermentation. The graph of FIG. 4 shows that IBA production for seven batch fermentations in corn mash using the same biodiesel volume for extraction achieved an average effective IBA titer of 72.1±5.4 g/L with no impact on production rate or partitioning capacity.

### Example 3. Recovery of IBA from Organic Solvent Extraction Using a Thin Film Evaporator

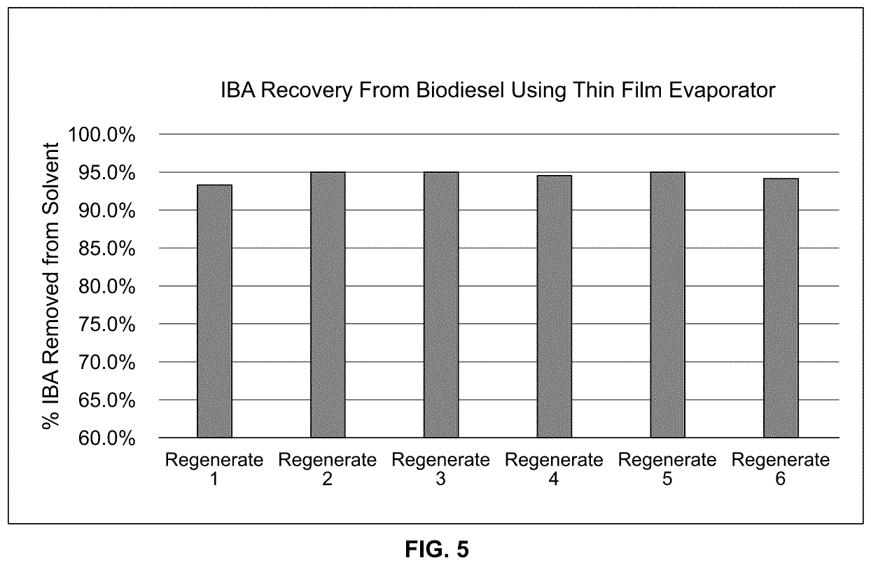

Either during or after the completion of the fermentation/extraction, the IBA may be recovered from the organic solvent for reuse in subsequent batch fermentations. A thin film evaporator (TFE) unit was acquired for use in demonstrating the efficiency of IBA recovery and the ability to demonstrate that the organic solvent may be reused over time to reduce operating expense. In this example, after completion of the fermentation, the organic solvent is separated from the aqueous broth and fed into the TFE at a prescribed flow rate, temperature and vacuum pressure. The IBA is separated from the solvent and collected as a distillate, and the residue (organic solvent) is collected and saved for reuse. Analysis shows that the TFE system removes 94-95% of the IBA from the solvent, and approximately 1 gram of IBA remained in the solvent after processing. The graph of FIG. 5 containing lines 41-46 (see Table 1) shows the consistency of IBA removed from biodiesel after six repeated fermentations (“regenerations”) using the same volume of solvent each time.

TABLE 1
Graph 4 Legend

| Line | 41 | 42 | 43 | 44 | 45 | 46 | 47 |
|---|---|---|---|---|---|---|---|
| Trial | Fresh | 1^{st} regen | 2^{nd} regen | 3^{rd} regen | 4^{th} regen | 5^{th} regen | 6^{th} regen |
| | biodiesel | | | | | | |

IBA recovery was also performed with petroleum diesel as the extracting solvent. Petroleum diesel was used under the same operating conditions described above for the biodiesel. The petrol diesel was separated and processed with the TFE under similar operating conditions. Table 2 below shows that 96-99% of the IBA present in the diesel was removed with approximately 0.1 gram of IBA remaining in the solvent.

TABLE 2
IBA Recovery with Petroleum Diesel as Extracting Solvent

| | Flask No. | | |
|---|---|---|---|
| | 1 | 2 | Average |
| | Initial Petro | Replicate Petro | Petro Diesel Test |
| Flask Name | Diesel Test | Diesel Test | Averages |
| Initial IBA Mass (grams) | 7.40352 g | 7.59808 g | 7.5008 g |
| Recovered IBA Mass (grams) | 7.090461 g | 7.346413 g | 7.218437 g |
| IBA Mass in Residue (grams) | 0 g | 0.2016 g | 0.1008 g |
| IBA Mass in Cold Trap Ice (grams) | 0 g | 0 g | 0 g |
| IBA Mass Balance | 95.8% | 99.3% | 97.6% |

The foregoing detailed description has been given for clearness of understanding only and no unnecessary limitations should be understood there from as modifications will be obvious to those skilled in the art.

While described in connection with specific embodiments thereof, it will be understood that the principles described herein are capable of further modifications and this application is intended to cover any variations, uses, or adaptations following, in general, the principles disclosed herein and including such departures from the present disclosure as coming within known or customary practice within the art to which the technology pertains and as may be applied to the essential features hereinbefore set forth and as follows in the scope of the appended claims.

The disclosures, including the claims, figures and/or drawings, of each and every patent, patent application, and publication cited herein are hereby incorporated herein by reference in their entireties.

### EXEMPLARY ASPECTS/EMBODIMENTS

Certain aspects, including embodiments/aspects of the present subject matter described above, may be beneficial alone or in combination, with one or more other aspects recited hereinbelow. In addition, while the present subject matter has been disclosed with reference to certain aspects recited below and in the claims, numerous modifications, alterations, and changes to the described aspects/embodiments are possible without departing from the sphere and scope of the present disclosure. Accordingly, it is intended that the present disclosure is not limited to the described embodiments, aspects, and claims, but that it has the full scope defined by the language of this disclosure and equivalents thereof. While the present technology has been described with reference to the specific aspects/embodiments thereof, it should be understood by those skilled in the art that various changes may be made, and equivalents may be substituted without departing from the true spirit and scope of the disclosure. In addition, many modifications may be made to adapt a particular situation, material, composition of matter, process, and/or process step or steps, to the objective, spirit and scope of the present disclosure. All such modifications are intended to be within the scope of the claims appended hereto.

Herein below are examples of aspects of the present technology.
A. A method of producing renewable alcohol, the method comprising:
   i. providing a recombinant yeast cell comprising an alcohol-producing metabolic pathway;
   ii. mixing the recombinant yeast cell with a source of glucose to produce a reaction mixture and fermenting the reaction mixture to produce alcohol;
   iii. contacting the reaction mixture and the alcohol with an extractant comprising diesel fuel, where the alcohol partitions into a non-aqueous organic extractant phase and the reaction mixture partitions into an aqueous phase; and
   iv. distilling the non-aqueous organic extractant phase to separate the alcohol.
B. The method according to paragraph A, where the reaction mixture and the alcohol are contacted with the extractant before the concentration of the alcohol reaches a level toxic to the recombinant yeast cell.
C. The method according to any one of paragraphs A and B, where the diesel fuel comprises fossil diesel, petroleum diesel, synthetic diesel, biodiesel, renewable diesel, hydrogenated oils, hydrogenated fats, or a combination thereof.
D. The method according to any one of paragraphs A-C, where the alcohol-producing metabolic pathway of step a) comprises an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.
E. The method according to any one of paragraphs A-D, where the recombinant yeast cell is selected from the group consisting of Saccharomyces, Kluyveromyces, Candida, Pichia, Issatchenkia, Debaryomyces, Hansenula, Yarrowia, Schizosaccharomyces, and combinations thereof.
F. The method according to any one of paragraphs A-E, where the renewable alcohol is selected from the group consisting of ethanol, 1-butanol, 2-butanol, isobutanol, tert-butanol, and combinations thereof.
G. The method according to any one of paragraphs A-F, where the extractant further comprises an organic solvent selected from the group consisting of phenetole, octanol, heptanol, oleyl alcohol, and mixtures thereof.
H. A method of producing a renewable fuel comprising converting the alcohol produced according to any one of paragraphs A-G to fuel.
I. A method of producing renewable fuel, the method comprising:
   i. providing a recombinant yeast cell comprising an alcohol-producing metabolic pathway;
   ii. mixing the recombinant yeast cell with a source of glucose to produce a reaction mixture and fermenting the reaction mixture to produce alcohol;
   iii. contacting the reaction mixture and the alcohol with an extractant comprising diesel fuel to produce a non-aqueous organic extractant phase and an aqueous phase, where the alcohol partitions into the non-aqueous organic extractant phase and the reaction mixture partitions into the aqueous phase;
   iv. separating the non-aqueous organic extractant phase to form the renewable fuel; and
   v. optionally, recycling the aqueous phase to a fermenter.
J. The method according to paragraph I, where the reaction mixture and the alcohol are contacted with the extractant before the concentration of the alcohol reaches a level toxic to the recombinant yeast cell.
K. The method according to any one of paragraphs I and J, where the diesel fuel comprises fossil diesel, petroleum diesel, synthetic diesel, biodiesel, renewable diesel, hydrogenated oils, hydrogenated fats, or a combination thereof.
L. The method according to any one of paragraphs I-K, where the alcohol producing metabolic pathway of step (a) comprises an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.
M. The method according to any one of paragraphs I-L, where the recombinant yeast cell is selected from the group consisting of Saccharomyces, Kluyveromyces, Candida, Pichia, Issatchenkia, Debaryomyces, Hansenula, Yarrowia, Schizosaccharomyces, and combinations thereof.
N. The method according to any one of paragraphs I-M, where the renewable alcohol is selected from the group consisting of ethanol, 1-butanol, 2-butanol, isobutanol, tert-butanol, and mixtures thereof.
O. The method according to any one of paragraphs I-N, where the extractant further comprises an organic solvent selected from the group consisting of phenetole, octanol, heptanol, oleyl alcohol, and mixtures thereof.
P. The method according to any one of paragraphs I-O, wherein the extractant is derived from fermenting feedstock.
Q. The method according to paragraph P, wherein the feedstock is corn oil R. A multiphase fermentation composition comprising:
   i. an aqueous phase comprising a recombinant yeast cell comprising an isobutanol-producing metabolic pathway and, optionally, a source of glucose; and
   ii. a non-aqueous organic extractant phase comprising isobutanol and diesel fuel.

What is claimed is:

1. A method of producing renewable alcohol, the method comprising:
a) providing a recombinant yeast cell comprising an alcohol-producing metabolic pathway;
b) mixing the recombinant yeast cell with a source of glucose to produce a reaction mixture and fermenting the first reaction mixture to produce alcohol;
c) contacting the reaction mixture and the alcohol with an extractant comprising diesel fuel, wherein the alcohol partitions into a non-aqueous organic extractant phase and the reaction mixture partitions into an aqueous phase; and
d) distilling the non-aqueous organic extractant phase to separate the alcohol.

2. The method according to claim 1, wherein the reaction mixture and the alcohol are contacted with the extractant before the concentration of the alcohol reaches a level toxic to the recombinant yeast cell.

3. The method according to claim 1, wherein the diesel fuel comprises fossil diesel, petroleum diesel, synthetic diesel, biodiesel, renewable diesel, hydrogenated oils, hydrogenated fats, or a combination thereof.

4. The method according to claim 1, wherein the alcohol producing metabolic pathway of step a) comprises an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.

5. The method according to claim 1, wherein the recombinant yeast cell is selected from the group consisting of Saccharomyces, Kluyveromyces, Candida, Pichia, Issatchenkia, Debaryomyces, Hansenula, Yarrowia, Schizosaccharomyces, and combinations thereof.

6. The method according to claim 1, wherein the renewable alcohol is selected from the group consisting of ethanol, 1-butanol, 2-butanol, isobutanol, tert-butanol, and combinations thereof.

7. The method according to claim 1, wherein the extractant further comprises an organic solvent selected from the group consisting of phenetole, octanol, heptanol, oleyl alcohol, and mixtures thereof.

8. A method of producing renewable fuel, the method comprising:
a) providing a recombinant yeast cell comprising an alcohol-producing metabolic pathway;
b) mixing the recombinant yeast cell with a source of glucose to produce a reaction mixture and fermenting the reaction mixture to produce alcohol;
c) contacting the reaction mixture and the alcohol with an extractant comprising diesel fuel to produce a non-aqueous organic extractant phase and an aqueous phase, wherein the alcohol partitions into the non-aqueous organic extractant phase and the reaction mixture partitions into the aqueous phase; and
d) separating the non-aqueous organic extractant phase to form the renewable fuel.

9. The method according to claim 8, further comprising recycling the aqueous phase to a fermenter.

10. The method according to claim 8, wherein the reaction mixture and the alcohol are contacted with the extractant comprising diesel fuel before the concentration of the alcohol reaches a level toxic to the recombinant yeast cell.

11. The method according to claim 8, wherein the diesel fuel comprises fossil diesel, petroleum diesel, synthetic diesel, biodiesel, renewable diesel, hydrogenated oils, hydrogenated fats, or a combination thereof.

12. The method according to claim 8, wherein the alcohol-producing metabolic pathway of step (a) comprises an enzyme selected from the group consisting of acetolactate synthase (ALS), ketol-acid reductoisomerase (KARI), dihydroxyacid dehydratase (DHAD), 2-keto-acid decarboxylase (KIVD), alcohol dehydrogenase (ADH), and combinations thereof.

13. The method according to claim 8, wherein the recombinant yeast cell is selected from the group consisting of Saccharomyces, Kluyveromyces, Candida, Pichia, Issatchenkia, Debaryomyces, Hansenula, Yarrowia, Schizosaccharomyces, and combinations thereof.

14. The method according to claim 8, wherein the renewable alcohol is selected from the group consisting of ethanol, 1-butanol, 2-butanol, isobutanol, tert-butanol, and mixtures thereof.

15. The method according to claim 8, wherein the extractant further comprises an organic solvent selected from the group consisting of phenetole, octanol, heptanol, oleyl alcohol, and mixtures thereof.

16. The method according to claim 8, wherein the extractant is derived from fermenting feedstock and modifying the feedstock to fatty acids and fatty acid methyl esters.

17. The method according to claim 16, wherein the feedstock is corn oil.

18. A multiphase fermentation composition comprising:
a) an aqueous phase comprising a recombinant yeast cell comprising an alcohol-producing metabolic pathway; and
b) a non-aqueous organic extractant phase comprising isobutanol and diesel fuel.

19. The method according to claim 18, wherein the aqueous phase further comprises a source of glucose.
